SEQUENCE LISTING (47 sequences)

SEQ ID NO: 1 - protein, 1954 aa, Homo sapiens
MRGPVGTEEELPRLFAEEMENEDEMSEEEDGGLEAFDDFFPVEPVSLPKKKKPKKLKENKCKGKRKKKEGSNDELSENEEDLEEKSESEGSDYSPNKKKKKKLKDKKEKKAKRKKKDEDEDDNDDGCLKEPKSSGQLMAEWGLDDVDYLFSEEDYHTLTNYKAFSQFLRPLIAKKNPKIPMSKMMTVLGAKWREFSANNPFKGSSAAAAAAAVAAAVETVTISPPLAVSPPQVPQPVPIRKAKTKEGKGPGVRKKIKGSKDGKKKGKGKKTAGLKFRFGGISNKRKKGSSSEEDEREESDFDSASIHSASVRSECSAALGKKSKRRRKKKRIDDGDGYETDHQDYCEVCQQGGEIILCDTCPRAYHLVCLDPELEKAPEGKWSCPHCEKEGIQWEPKDDDDEEEEGGCEEEEDDHMEFCRVCKDGGELLCCDACPSSYHLHCLNPPLPEIPNGEWLCPRCTCPPLKGKVQRILHWRWTEPPAPFMVGLPGPDVEPSLPPPKPLEGIPEREFFVKWAGLSYWHCSWVKELQLELYHTVMYRNYQRKNDMDEPPPFDYGSGDEDGKSEKRKNKDPLYAKMEERFYRYGIKPEWMMIHRILNHSFDKKGDVHYLIKWKDLPYDQCTWEIDDIDIPYYDNLKQAYWGHRELMLGEDTRLPKRLLKKGKKLRDDKQEKPPDTPIVDPTVKFDKQPWYIDSTGGTLHPYQLEGLNWLRFSWAQGTDTILADEMGLGKTVQTIVFLYSLYKEGHSKGPYLVSAPLSTIINWEREFEMWAPDFYVVTYTGDKESRSVIRENEFSFEDNAIRSGKKVFRMKKEVQIKFHVLLTSYELITIDQAILGSIEWACLVVDEAHRLKNNQSKFFRVLNSYKIDYKLLLTGTPLQNNLEELFHLLNFLTPERFNNLEGFLEEFADISKEDQIKKLHDLLGPHMLRRLKADVFKNMPAKTELIVRVELSQMQKKYYKFILTRNFEALNSKGGGNQVSLLNIMMDLKKCCNHPYLFPVAAVEAPVLPNGSYDGSSLVKSSGKLMLLQKMLKKLRDEGHRVLIFSQMTKMLDLLEDFLEYEGYKYERIDGGITGGLRQEAIDRFNAPGAQQFCFLLSTRAGGLGINLATADTVIIYDSDWNPHNDIQAFSRAHRIGQNKKVMIYRFVTRASVEERITQVAKRKMMLTHLVVRPGLGSKSGSMTKQELDDILKFGTEELFKDDVEGMMSQGQRPVTPIPDVQSSKGGNLAASAKKKHGSTPPGDNKDVEDSSVIHYDDAAISKLLDRNQDATDDTELQNMNEYLSSFKVAQYVVREEDGVEEVEREIIKQEENVDPDYWEKLLRHHYEQQQEDLARNLGKGKRIRKQVNYNDASQEDQEWQDELSDNQSEYSIGSEDEDEDFEERPEGQSGRRQSRRQLKSDRDKPLPPLLARVGGNIEVLGFNARQRKAFLNAIMRWGMPPQDAFNSHWLVRDLRGKSEKEFRAYVSLFMRHLCEPGADGAETFADGVPREGLSRQHVLTRIGVMSLVRKKVQEFEHVNGKYSTPDLIPEGPEGKKSGEVISSDPNTPVPASPAHLLPAPLGLPDKMEAQLGYMDEKDPGAQKPRQPLEVQALPAALDRVESEDKHESPASKERAREERPEETEKAPPSPEQLPREEVLPEKEKILDKLELSLIHSRGDSSELRPDDTKAEEKEPIETQQNGDKEEDDEGKKEDKKGKFKFMFNIADGGFTELHTLWQNEERAAVSSGKIYDIWHRRHDYWLLAGIVTHGYARWQDIQNDPRYMILNEPFKSEVHKGNYLEMKNKFLARRFKLLEQALVIEEQLRRAAYLNMTQDPNHPAMALNARLAEVECLAESHQHLSKESLAGNKPANAVLHKVLNQLEELLSDMKADVTRLPSMLSRIPPVAARLQMSERSILSRLTNRAGDPTIQQGAFGSSQMYSNNFGPNFRGPGPGGIVNYNQMPLGPYVTDI

SEQ ID NO: 2 - DNA, 21 bp, Artificial Sequence
  Description of Artificial Sequence Synthetic primer
cctcatggactaattatggac

SEQ ID NO: 3 - DNA, 20 bp, Artificial Sequence
  Description of Artificial Sequence Synthetic primer
ccagtttcactaatgacaca

SEQ ID NO: 4 - DNA, 24 bp, Artificial Sequence
  Description of Artificial Sequence Synthetic primer
aactggctgagtgacgccctttat

SEQ ID NO: 5 - DNA, 24 bp, Artificial Sequence
  Description of Artificial Sequence Synthetic primer
tgagacgtgctacttccatttgtc

SEQ ID NO: 6 - DNA, 25 bp, Artificial Sequence
  Description of Artificial Sequence Synthetic primer
tggaatcctgtggcatccatgaaac

SEQ ID NO: 7 - DNA, 25 bp, Artificial Sequence
  Description of Artificial Sequence Synthetic primer
taaaacgcagctcagtaacagtccg

SEQ ID NO: 8 - DNA, 21 bp, Artificial Sequence
  Description of Artificial Sequence Synthetic primer
tatggatccactctccagaaa

SEQ ID NO: 9 - DNA, 20 bp, Artificial Sequence
  Description of Artificial Sequence Synthetic primer
caaagtctcctccaaggctg

SEQ ID NO: 10 - DNA, 21 bp, Artificial Sequence
  Description of Artificial Sequence Synthetic oligonucleotide
gccctccttgcattatgctaa

SEQ ID NO: 11 - DNA, 21 bp, Artificial Sequence
  Description of Artificial Sequence Synthetic oligonucleotide
cggaagagcgtgagtacaaat

SEQ ID NO: 12 - DNA, 21 bp, Artificial Sequence
  Description of Artificial Sequence Synthetic oligonucleotide
ggcaataaaccctacagcaaa

SEQ ID NO: 13 - DNA, 21 bp, Artificial Sequence
  Description of Artificial Sequence Synthetic oligonucleotide
gccctttatcactgggataaa

SEQ ID NO: 14 - DNA, 21 bp, Artificial Sequence
  Description of Artificial Sequence Synthetic oligonucleotide
ggaagagtacttggattataa

SEQ ID NO: 15 - DNA, 21 bp, Artificial Sequence
  Description of Artificial Sequence Synthetic oligonucleotide
tgcccacaggcctgcctttca

SEQ ID NO: 16 - DNA, 21 bp, Artificial Sequence
  Description of Artificial Sequence Synthetic oligonucleotide
ggcaccacaacctgcatcaaa

SEQ ID NO: 17 - DNA, 21 bp, Artificial Sequence
  Description of Artificial Sequence Synthetic oligonucleotide
ggagctgagtgttaagtacat

SEQ ID NO: 18 - DNA, 21 bp, Artificial Sequence
  Description of Artificial Sequence Synthetic oligonucleotide
ccgagatacatgatcctaaat

SEQ ID NO: 19 - DNA, 21 bp, Artificial Sequence
  Description of Artificial Sequence Synthetic oligonucleotide
cgcaagcaggtcaactacaat

SEQ ID NO: 20 - DNA, 21 bp, Artificial Sequence
  Description of Artificial Sequence Synthetic oligonucleotide
cgagagcctaagccagctgaa

SEQ ID NO: 21 - DNA, 21 bp, Artificial Sequence
  Description of Artificial Sequence Synthetic oligonucleotide
ccgctcttcgaagttggagaa

SEQ ID NO: 22 - DNA, 21 bp, Artificial Sequence
  Description of Artificial Sequence Synthetic oligonucleotide
cctcagcaaatattacctgaa

SEQ ID NO: 23 - DNA, 21 bp, Artificial Sequence
  Description of Artificial Sequence Synthetic oligonucleotide
ggcgacacaaaggtgtgagaa

SEQ ID NO: 24 - DNA, 21 bp, Artificial Sequence
  Description of Artificial Sequence Synthetic oligonucleotide
ccgcagattgagattaataaa

SEQ ID NO: 25 - DNA, 21 bp, Artificial Sequence
  Description of Artificial Sequence Synthetic oligonucleotide
cggcggcggctccattaactt

SEQ ID NO: 26 - DNA, 21 bp, Artificial Sequence
  Description of Artificial Sequence Synthetic oligonucleotide
gccatccttatccagagcaaa

SEQ ID NO: 27 - DNA, 21 bp, Artificial Sequence
  Description of Artificial Sequence Synthetic oligonucleotide
ccaagtaatgtgaatgctaat

SEQ ID NO: 28 - DNA, 21 bp, Artificial Sequence
  Description of Artificial Sequence Synthetic oligonucleotide
gctctcatcccgggtctgtat

SEQ ID NO: 29 - DNA, 21 bp, Artificial Sequence
  Description of Artificial Sequence Synthetic oligonucleotide
agaatttatctgagtcgccta

SEQ ID NO: 30 - DNA, 21 bp, Artificial Sequence
  Description of Artificial Sequence Synthetic oligonucleotide
ggccagcaaagccagactata

SEQ ID NO: 31 - DNA, 21 bp, Artificial Sequence
  Description of Artificial Sequence Synthetic oligonucleotide
ggttagctcaacgcattatta

SEQ ID NO: 32 - DNA, 20 bp, Artificial Sequence
  Description of Artificial Sequence Synthetic primer
gatctggcaccacaccttct

SEQ ID NO: 33 - DNA, 20 bp, Artificial Sequence
  Description of Artificial Sequence Synthetic primer
ggggtgttgaaggtctcaaa

SEQ ID NO: 34 - DNA, 20 bp, Artificial Sequence
  Description of Artificial Sequence Synthetic primer
ttgcactctggtgtctgagc

SEQ ID NO: 35 - DNA, 20 bp, Artificial Sequence
  Description of Artificial Sequence Synthetic primer
tctgcgcttggagtgataga

SEQ ID NO: 36 - DNA, 20 bp, Artificial Sequence
  Description of Artificial Sequence Synthetic primer
tgaatcctccccttccatca

SEQ ID NO: 37 - DNA, 20 bp, Artificial Sequence
  Description of Artificial Sequence Synthetic primer
tctcacgaagggtccagcat

SEQ ID NO: 38 - DNA, 20 bp, Artificial Sequence
  Description of Artificial Sequence Synthetic primer
agcgcgaagagaggagacta

SEQ ID NO: 39 - DNA, 20 bp, Artificial Sequence
  Description of Artificial Sequence Synthetic primer
cacttccggacacatcttca

SEQ ID NO: 40 - DNA, 20 bp, Artificial Sequence
  Description of Artificial Sequence Synthetic primer
gagagccaccagcacctatc

SEQ ID NO: 41 - DNA, 20 bp, Artificial Sequence
  Description of Artificial Sequence Synthetic primer
tgctttcatgtcactcagca

SEQ ID NO: 42 - DNA, 18 bp, Artificial Sequence
  Description of Artificial Sequence Synthetic primer
cgctgctccgatggtgat

SEQ ID NO: 43 - DNA, 22 bp, Artificial Sequence
  Description of Artificial Sequence Synthetic primer
tcgggatacaaatttccttcca

SEQ ID NO: 44 - DNA, 19 bp, Artificial Sequence
  Description of Artificial Sequence Synthetic primer
gtcacacgactgggcgatt

SEQ ID NO: 45 - DNA, 19 bp, Artificial Sequence
  Description of Artificial Sequence Synthetic primer
catgctgctccagatggct

SEQ ID NO: 46 - DNA, 23 bp, Artificial Sequence
  Description of Artificial Sequence Synthetic primer
tgaggctagagaggatcttgaga

SEQ ID NO: 47 - DNA, 21 bp, Artificial Sequence
  Description of Artificial Sequence Synthetic primer
gcagaagagctgctacgtgaa

(19) United States
(12) Patent Application Publication
(10) Pub. No.: US 2011/0055941 A1
(43) Pub. Date: Mar. 3, 2011

(54) Chd5 is a novel tumor suppressor gene

(75) Inventors: Alea A. Mills, Lloyd Harbor, NY (US); Anindya Bagchi, Huntington, NY (US); Cristian Constantin Papazoglu-Statescu, Forest Hills, NY (US)

(73) Assignee: COLD SPRING HARBOR LABORATORY, Cold Spring Harbor, NY (US)

(21) Appl. No.: 11/893,721

(22) Filed: Aug. 16, 2007

Related U.S. Application Data
(60) Provisional application No. 60/838,508, filed on Aug. 16, 2006.

(51) Int. Cl. A01K 67/027; C12N 5/02; C12N 5/07; C12N 5/071; G01N 33/53; C12N 5/10; C12Q 1/02

(52) U.S. Cl. 800/18; 435/375; 435/354; 435/366; 435/363; 435/372; 435/7.1; 435/325; 435/29

(57) ABSTRACT

The invention provides methods of treating cancer using a Chd5 protein or an agonist thereof Also provided are diagnostics, screening methods of cancer therapeutics, and cancer models useful for studying cancer biology and drug screening.

This application claims priority from U.S. Provisional Application 60/838,508, filed Aug. 16, 2006. 35 U.S.C. §119(e).

This invention was supported in part by Developmental Funds, Cancer Center Support Grant No. CA45508 from the National Cancer Institute. The United States Government may have certain rights in this invention.

### BACKGROUND OF THE INVENTION

Identifying cancer genes and understanding how they contribute to tumorigenesis are critical steps in controlling cancer. Although progress has been made with oncogenes, success has been limited with tumor suppressors. Most tumor suppressor genes identified thus far were found using positional cloning of genes involved in human hereditary cancer susceptibility syndromes, including retinoblastoma (Friend et al., 1986, Nature 323, 643-646; Lee et al., 1987, Science 235, 1394-1399), Wilm's tumor (Call et al., 1990, Cell 60, 509-520; Gessler et al., 1990, Nature 343, 774-778), neurofibromatosis type I (Cawthon et al., 1990, Cell 62, 193-201; Wallace et al., 1990, Science 249, 181-189), colorectal cancer (Fearon et al., 1990, Science 247, 49-56; Fishel et al., 1993, Cell 75, 1027-1038; Groden et al., 1991, Cell 66, 589-600; Kinzler et al., 1991, Science 251, 1366-1370), and breast cancer (Miki et al., 1994, Science 266, 66-71; Wooster et al., 1995 Nature 378, 789-792). 71; Wooster et al., 1995 Nature 378, 789-792). However, most cancers involve spontaneous mutations, therefore key tumor suppressors have likely eluded detection using these classical approaches.

More recently, systematic approaches have been used to identify cancer genes by scanning the genome for chromosomal gains and losses. Since end-stage tumors are typically used in these studies, a complication of this approach is the inability to determine which genetic alterations were involved in the initiation of the tumor and which are simply consequences of the tumorigenic process. In addition, genome-wide approaches frequently identify a plethora of genomic alterations, making it difficult to identify events that initiate the tumorigenic process.

Despite these challenges, molecular analyses of sporadic cancers have identified regions that are very commonly deleted, adding weight to the hypothesis that genes in these intervals encode proteins that protect from malignancy. The short arm of human chromosome 1 (1p), for example, is frequently deleted in human cancer, with 1p36 deletion being the most common lesion. 1p36 deletions were first reported in neuroblastoma in 1977 (Brodeur et al., 1977, Cancer 40, 2256-2263) and have since been confirmed by others (Caron et al., 2001, Cancer 30, 168-174; Godfried et al., 2002, Eur J Cancer 38, 1513-1519; White et al., 1995, Proc Natl Acad Sci USA 92, 5520-5524; White et al., 2005, Oncogene 24, 2684-2694). Other neural-related malignancies associated with 1p36 deletions include meningioma (Piaskowski et al., 2005, Cancer Genet Cytogenet 162, 63-67), melanoma (Dracopoli et al., 1989, Proc Natl Acad Sci USA 86, 4614-4618; Poetsch et al., 2003, Res 13, 29-33), pheochromocytoma (Aarts et al., 2006, Genes Chromosomes Cancer 45, 83-93; Moley et al., 1992, Cancer Res 52, 770-774; Mulligan et al., 1993, Genes Chromosomes Cancer 6, 166-177), and oligodendroglioma (Barbashina et al., 2005, Clin Cancer Res 11, 1119-1128; Bello et al., 1995, Int J Cancer 64, 207-210; Dong et al., 2004, Br J Cancer 91, 1105-1111; Felsberg et al., 2004, Brain Pathol 14, 121-130). In addition, 1p36 is deleted in hematopoetic malignancies including acute myelogenous leukemia (AML) (Mori et al., 2003, Br J Haematol 122, 226-230), chronic myelogenous leukemia (CML) (Mori et al., 1998, Blood 92, 3405-3409) and non-Hodgkin's lymphoma (Melendez et al., 2003, Leuk Res 27, 627-633), as well as in a variety of epithelial malignancies including those of the thyroid (Kleer et al., 2000, Endocr Pathol 11, 137-143), stomach (Ezaki et al., 1996, Br J Cancer 73, 424-428; Wada et al., 1988, Cancer Res 48, 2988-2992), colon (Leister et al., 1990, Cancer Res 50, 7232-7235; Praml et al., 1995, Oncogene 11, 1357-1362; Zhou et al., 2004, World J Gastroenterol 10, 1431-1435), cervix (Cheung et al., 2005, Gynecol Oncol 96, 510-515), and breast (Bieche et al., 1993, Cancer Research 53, 1990-1994; Bieche et al., 1999, Genes Chromosomes Cancer 24, 255-263; Hoggard et al., 1995, Genes, Chromosomes & Cancer 12, 24-31; Nagai et al., 1995, Cancer Research 55, 1752-1757). These data suggest that one or more tumor suppressor genes mapping to 1p36 are lost or inactivated in a variety of human cancers. Identification of the gene mapping to 1p36 and gaining insight into how its encoded protein normally functions to suppress malignancy may reveal an underlying molecular mechanism that drives tumorigenesis in diverse types of human cancers when inactivated. Although a number of laboratories have focused on identifying these genes, the 1p36 tumor suppressor has remained elusive for three decades.

### SUMMARY OF THE INVENTION

This invention is based on our discovery that the chromodomain helicase DNA binding domain 5 protein (Chd5) is a tumor suppressor encoded by a gene within 1p36. This invention provides methods of (1) inhibiting growth of a mammalian cell, (2) inducing apoptosis of a mammalian cell, and/or (3) inducing expression of p16^{Ink4a}, p19^{Arf}, or p53 in a mammalian cell, comprising increasing the level of a Chd5 protein within the cell. To increase the level of the Chd5 protein in the cell, one can introduce to the cell a Chd5 protein (e.g., by contacting the cell with the Chd5 protein or an expression vector such as a viral vector encoding the Chd5 protein). The mammalian cell can be a human, mouse, or nonhuman primate cell. It can be a cancer cell, such as a human breast cancer cell, a human lymphoma cell, a human glioma cell, a human squamous cancer cell, a human sarcoma cell, or a human kibernoma cell. In some embodiments, the mammalian cell has a deletion in the endogenous Chd5 allele.

This invention provides a method of treating cancer in a subject (e.g., human or mouse), comprising administering to the subject a composition that increases the Chd5 level in the subject (e.g., a composition comprising an effective amount of a Chd5 protein or an expression vector thereof, or of an agent that activates the endogenous expression of Chd5, as well as a pharmaceutically acceptable carrier). In some embodiments, the Chd5 protein is provided in form of an expression vector (e.g., an adenoviral, lentiviral, or retroviral vector) encoding the protein. The treatment will prevent tumorigenesis, tumor progression (e.g., metastasis), and tumor recurrence. The treatment will also induce tumor regression.

The invention also provides a method of increasing growth of a mammalian cell (including a stem cell), comprising increasing the Chd5 level in the cell, by, e.g., introducing into the cell an antisense or interfering RNA against a Chd5 transcript. The RNA can be introduced in form of an DNA expression vector encoding the RNA.

The invention further provides a method of diagnosing cancer in a human subject, comprising: providing a DNA sample from a tissue of the patient, and detecting, in said DNA sample, deletion of a nucleic acid sequence (e.g., at least 20, 50, 100, or 1,000 nucleotides in length) (1) in a Chd5 gene or (2) flanking the Chd5 gene in the D4Mit190-D4Mit51 interval in chromosome region 1p36, wherein said deletion indicates that the patient has, or is susceptible of developing, cancer in said tissue.

The invention further provides a method of diagnosing cancer in a human patient, comprising: providing a tissue sample from the patient, determining the level of a Chd5 protein in said tissue sample, comparing said level of said Chd5 protein in the patient sample with a level of said Chd5 protein in a control sample, wherein a lower level of said Chd5 protein in said patient sample as compared to the control sample is indicative of the presence of cancer or susceptibility to cancer.

The invention provides mice some or all of whose cells comprise a genome comprising an inactivating mutation in a Chd5 gene, and optionally comprising an activated oncogene. The invention further provides mammalian cells with these genetic features.

The invention also provides mice some or all of whose cells comprise a genome comprising a coding sequence for an antisense or interfering RNA against a Chd5 transcript, and optionally comprising an activated oncogene. In some embodiments, the coding sequence for the RNA is under the transcriptional control of an inducible promoter. The invention also provides mammalian cells with these genetic features.

The invention also provides methods of screening for anti-cancer compounds. Such compounds can activate the endogenous expression of Chd5 or agonize the activity of Chd5 in a mammalian cell. The invention also provides methods of screening for stem cell-promoting compounds (i.e., compounds that maintain stem cell pluripotency and increase their proliferation). Such compounds can inactivate the endogenous expression of Chd5 or antagonize the activity of Chd5 in a stem cell.

### BRIEF DESCRIPTION OF THE DRAWINGS

FIGS. 1A and 1B are a panel of diagrams showing the genes mapping to human 1p36 correspond to mouse. chromosome 4. A. Conserved linkage between human 1p36 and distal mouse chromosome 4 (hatched bars). An expanded view of this region depicts cytogenetic bands (right). The 4.3 megabase (Mb) region between endpoints D4Mit190 and D4Mit51 is the focus of the current study. B. The D4Mit190-D4Mit51 region contains the genes shown in clusters I-IV (see Table 1, infra, for details).

FIGS. 2A-D are a panel of diagrams and Southern blot images showing engineering mouse strains with chromosome deletions and duplications of the D4Mit190 and D4Mit51 interval. A. Modification of endpoint D4Mit190 by gene targeting in ES cells. Gene targeting using the insertion vector shown was used to integrate a loxP site (triangle), a neomycin resistance cassette (N), the 5′ half of the hprt locus (5′) and the Tyrosinase gene (Ty) at the endogenous D4Mit190 locus (upper panel). Accurate targeting of G418-resistant (G^{R}) clones was assessed by Southern analysis of NdeI (Nd)-digested DNA using the 1.5 kb gap probe; a 16.2 kb fragment is diagnostic for singly-targeted (st) clones (lower panel). B. A second gene targeting step in the st clone shown in A was performed to modify endpoint D4Mit51. The loxP site, a puromycin resistance cassette (P), the 3′ half of the hprt locus (3′), and an Agouti transgene (Ag) were integrated at the D4Mit51 locus (upper panel). Accurate targeting of puromycin-resistant (P^{R}) clones was assessed by Southern analysis of SpeI (Sp)-digested DNA using the 1.3 kb gap probe; a 5.2 kb fragment is diagnostic for doubly-targeted (dt) clones (lower panel). C. Cre-mediated recombination in doubly targeted clones. Clones that had loxP sites and resistance cassettes integrated in Cis (on the same chromosome homolog) generated deficiency (df) and duplication (dp) alleles within the same ES cell and were identified by their resistance to G418, puromycin, and hypoxanthine aminopterin thymidine (HAT). D. dfldp clones were used to generate chimeric mice; PCR genotyping of late stage embryos from dfldp chimeras and wild type matings identifies +/+ (lanes 1, 6), dfl+ (lanes 4, 5, 7) and dp/+ (lanes 2, 3) progeny. M, λBstEI marker; d, water control. Drug resistance that is gained in a particular step is underlined.

FIGS. 3A-E are a panel of graphs and photographs showing the phenotypic characterization of dfl+ and dp/+ mefs. A. Equivalent numbers of +/+ (black), dp/+ (blue), and dfl+ (red) mefs were plated in triplicate and counted at 2-day intervals. B. Flow cytometric analysis of +/+ (left), dp/+ (middle), and dfl+ (right) mefs. C. Senescence-associated-beta-galactosidase (SA-β-gal) assay in +/+, dfl+ (not shown), and dp/+ mefs (left); the percentage of SA-β-gal-positive cells (+/+, dfl+, and dp/+ mefs) is quantitated (right). D. Mefs were counted and serially passaged at 3-day intervals to evaluate cellular lifespan (upper panel). PCR analysis using primers specific for the df allele (df), primers that differentiate between the +/+ (B6 mouse strain)—and the df (129Sv mouse strain) chromosomes (D4Mit308), and control primers (actin) were used to genotype and to detect retention of the wild type chromosome in immortalized dfl+ cells. Genomic DNA from the 129Sv strain (lane 2), the B6 strain (lane 3), F1 (B6: 129Sv) dp/+ mice (lane 4), and passage 37 (i.e., immortalized) dp/+ cells from F1 mice (lane 5); water control (lane 1). E. Foci formation assay of dfl+ cells.

FIGS. 4A-C are a panel of photographs showing the developmental defects and enhanced apoptosis in dp/+ mice. A. Whole mount analysis of E15.5 embryos obtained from dfldp chimeras and +/+ crosses reveal developmental defects in dp/+ embryos. B. Histological analysis of dp/+ embryos at E15.5 identifies clusters of cells that appear apoptotic. C. TUNEL assay labels apoptotic cells within the developing eye and neural tube of dp/+ mice.

FIGS. 5A and 5B are a panel of photographs and graph showing that reducing dosage of the D4Mit190-D4Mit51 interval genetically rescues the dp/+ phenotype. A. The df allele rescues the dp/+ phenotype in vivo. Whole mount analysis (upper panel) and genotyping by PCR (lower panel) of E17.5 embryos from dfldp chimeras times dfl+ matings identifies dp/+ progeny with developmental defects and dfldp progeny that are developmentally normal. B. The compromised proliferation of dp/+ mefs in culture is restored to wild type by reducing dosage of the D4Mit190 and D4Mit51 interval.

FIGS. 6A-C are a panel of photographs and graphs showing that dosage of the D4Mit190-D4Mit51 interval directly correlates with p53 function. A. RNAi-mediated knockdown of p53 rescues the compromised proliferation of dp/+ cells in culture. dp/+ mefs infected with a retroviral construct expressing either GFP or shp53 (left) were assayed for proliferation (right). B. p53 deficiency rescues the excessive apoptosis of dp/+ mice in vivo. dp/+ mice were established in p53-compromised backgrounds. Representative images of litters including dp/+; p53+/− (black asterisks) and Southern analysis (lower left panel) demonstrate that p53 heterozygosity partially rescues the excessive apoptosis and developmental defects of dp/+ mice. Generation of dp/+; p53−/− mice (lower middle) were identified by Southern and PCR (lower right, red asterisks), demonstrating that p53 nullizygosity rescues the apoptotic phenotype of dp/+ mice. C. Heterozygous D4Mit190-D4Mit51 deficiency compromises the expression—and transcriptional activity of p53. p53 expression—and transcriptional activation of p53 target genes in +/+ and dfl+ cells was evaluated at the transcript level by real-time PCR (left). p53 protein levels were assessed by Western analysis (right) in +/+ and dfl+ cells prior to—and following adriamycin treatment. Long—(upper panel) and short—(middle panel) exposures were used to assess p53 protein levels; actin expression (lower panel) serves as loading control.

FIGS. 7A-C are a panel of photographs and bar graphs showing that RNAi-mediated knockdown identifies Chd5 as the D4Mit190-D4Mit51 proliferation-suppressing gene. A. RNAi-mediated knockdown of specific target genes in +/+ mefs assessed by real-time PCR. GFP-expressing cells serve as reference. B. Knockdown of Chd5 using two different short hairpins (shChd5-2, and shChd5-4) rescues the proliferation defect of dp/+ mefs, whereas knockdown of Kcnabb or Dnajc has no effect on proliferation. The phenotype of cells 3 days after infection (left) and proliferation 5 days after infection (right) are shown. C. Expression of Chd5, p53, and the p53 target genes p21 and Mdm-2 in +/+-GFP-, dfl+-, +/+-shChd5-, and +/+-shp53 mefs was assessed by real-time PCR.

FIGS. 8A-D are a panel of photographs and graphs showing that Chd5 functions as a tumor suppressor. A. Ras induces proliferation of dfl+ and Chd5 deficient cells. +/+-GFP-, +/+-shDnajc-, dfl+-, +/+-shChd5-, and +/+-shp53 mefs were infected with RasV12-expressing retrovirus and the phenotype—(upper panel) and proliferation—(lower panel) analyzed. B. Ras induces transformation of dfl+ and Chd5 deficient cells. Transformation was assessed by staining colonies with crystal violet (upper panels) in +/+-Ras, +/+-shp53-Ras, and dfl+ mefs and by soft agar assays where foci were counted—(middle panel) and assessed for morphology—(lower panel) in +/+-GFP-, dfl+-, +/+-shChd5-, and +/+-shp53 mefs. C. Expression of p53, p16^{Ink4a} and p19^{Arf} are compromised in both dfl+ and +/+-shChd5-2 mefs. Real-time PCR prior to—(upper panel) and following—(middle panel) expression of Ras was used to evaluate expression of p16^{Ink4a} and p19^{Arf}. Western analysis in Ras-infected cells was used to assess expression of Ras, p53, p16^{Ink4a} and p19^{Arf}; actin was used as a loading control (lower panel). D. Deficiency of the D4Mit190-D4Mit51 interval predisposes to spontaneous tumorigenesis. Histological—(upper panel) and Southern—(lower panel) analyses of a squamous cell carcinoma of the skin that developed spontaneously in a 10-month old dfl+ mouse were performed. FIG. 9: Model for tumor suppression mediated by Chd5. Normal cells are diploid for the Chd5 locus and maintain p53's tumor suppressive function by appropriate regulation of p19^{Arf}, an inhibitor of the p53-degrading enzyme Mdm-2 (upper). Duplication of the D4Mit190-D4Mit51 region increases dosage of Chd5, which enhances p53-mediated pathways, presumably by enhancing expression of p19^{Arf} (middle). This enhanced p53 activity triggers the potent tumor suppressive mechanisms of senescence and apoptosis, both of which are rescued by p53 deficiency. In contrast, heterozygous deficiency of the D4Mit190-D4Mit51 region decreases dosage of Chd5, which compromises p53 function by reducing expression of p19^{Arf} (lower). This compromise in p53's tumor suppressive function predisposes to malignancy.

FIG. 9 is a schematic diagram showing Chd5's tumor suppression functions.

FIG. 10 is a schematic diagram showing the strategy for Chd5 gene targeting deletion in a mouse using MICER.

FIGS. 11A and 11B are bar diagrams respectively showing the analysis of fetal livers (A) and brains (B) from mice with altered Chd5 dosage (n=6).

### DETAILED DESCRIPTION OF THE INVENTION

This invention is based on the discovery that the chromodomain helicase DNA binding domain 5 protein (Chd5) is a tumor suppressor that controls proliferation, apoptosis, and senescence. Chd5 does so by modulation of p53 through regulation of the p16^{Ink4a}/p19^{Arf} locus.

Chromatin remodeling is a mechanism by which gene expression is regulated. Chromatin structure is controlled at least in part by post-translational modification of histones, as well as by chromodomain proteins. Chd5 is a protein with chromatin remodeling, helicase and DNA-binding motifs. It is the fifth member of the human Chd gene family. This gene is homologous to Chd3 and Chd4, which encode proteins that are part of the nucleosome remodeling and histone deacetylation (NuRD) complex. Chd5 is expressed in a variety of tissues and cells, including epidermis of the skin, esophagus, uterus, bladder, spleen, brain, fetal brain, cerebellum, adrenal gland, and embryonic fibroblasts.

The Chd5 gene is located in human chromosome 1p36. Rearrangements of 1p were first discovered in 1977, when Brodeur and colleagues reported deficiency of this region in neuroblastoma (Brodeur et al., supra). Since that time, numerous studies revealed that 1p36 deletions are extremely common in end-stage tumors of epithelial-, neural-, and hematopoietic origin (see above). A number of groups had been striving to discover tumor suppressors mapping to 1p36, but this gene remained elusive.

Previously, we confirmed the existence of a novel tumor suppressor mapping to 1p36 using chromosome engineering (Mills et al., 2001, Trends in Genetics 17, 331-339; Yu et al., 2001, Nat Rev Genet 2, 780-790). This functional genomics approach involved the generation of mouse models that mimic spontaneous human cancers. This was accomplished by establishing mouse strains with gains and losses of genomic intervals spanning the region corresponding to human 1p36. Using this approach, we have identified a 4.3 megabase (Mb) region that is a potent regulator of proliferation, cellular senescence, apoptosis, and tumorigenesis.

Using mouse models with specific chromosome rearrangements of the corresponding region of mouse chromosome 4, we predicted that deletions encompassing a potent tumor suppressor locus would compromise tumor suppressive mechanisms and predispose to cancer, thereby functionally pinpointing novel tumor suppressors to a defined region of the genome. In addition to generating these loss-of-function models, the chromosome engineering approach was also used to generate gain-of-function models of the same genomic interval, allowing us to directly compare the phenotype caused by decreased—and increased dosage, respectively.

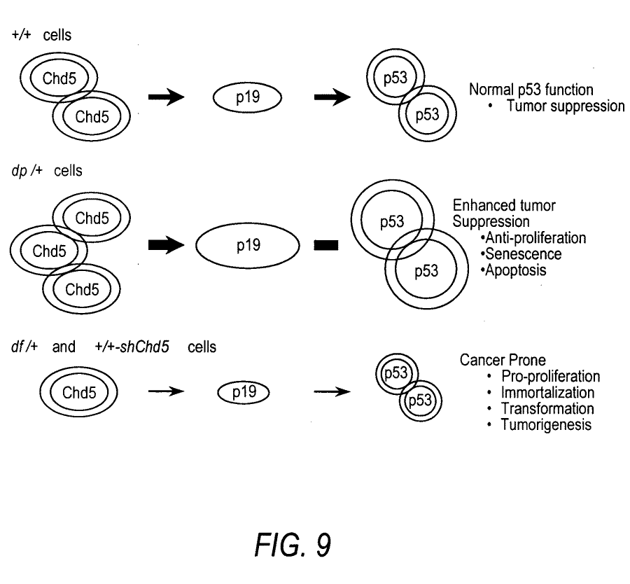

We have made several major findings. First, we identified the D4Mit190-D4Mit51 region as a potent regulator of proliferation, senescence and apoptosis. We found an inverse correlation between dosage of this region and cellular proliferation. While increased dosage triggers the tumor suppressive mechanisms of cellular senescence and apoptosis, decreased dosage of the same region enhances immortalization, increases spontaneous foci formation, and renders cells sensitive to oncogenic transformation. Second, we demonstrated that the D4Mit190-D4Mit51 region positively regulates p53 via p19^{Arf}. We found that the enhanced senescence and apoptosis caused by increased dosage of the D4Mit190-D4Mit51 region is p53-dependent, both in cultured cells and in vivo, demonstrating that p53 function is exacerbated by increased dosage of this region. On the other hand, p53 function is severely compromised by heterozygosity of the D4Mit190-D4Mit51 region, thereby facilitating oncogenic transformation in cultured cells and predisposing to spontaneous tumorigenesis in vivo. We demonstrated that deficiency of the D4Mit190-D4Mit51 region also cripples both basal—and oncogene-induced expression of p16^{Ink4a}/p19^{Arf}, suggesting a mechanism for both compromised p53 function and susceptibility to transformation. Third, we identified Chd5 as the gene within the D4Mit190-D4Mit51 region that regulates cellular proliferation and mediates tumor suppressive mechanisms. Indeed, depletion of Chd5 rescued the proliferative defect of cells with increased dosage of the D4Mit190-D4Mit51 region, indicating that enhanced expression of Chd5 alone is responsible for the proliferative defect of these cells. Most importantly, wild type cells in which Chd5 had been specifically knocked down by RNAi phenocopy cells with an engineered deletion of the D4Mit190-D4Mit51 region. Both showed enhanced proliferation, sensitivity to oncogenic transformation, and compromised expression of p16^{Ink4a}, p19^{Arf}, and p53. Furthermore, we found that CHD5, the human counterpart of mouse Chd5, had decreased expression levels in human cancers. These findings (schematized in FIG. 9) functionally identify Chd5 as a novel tumor suppressor mapping to human 1p36.

The discovery that Chd5 is genetically linked to the Ink4/Arf tumor suppression pathway provides the molecular basis for the overlap between the types of tumors caused by Bmi1 mutations and the types of tumors caused by Chd5 deletions. Bmi1 is an oncogene that has been shown to promote tumorigenesis by crippling the Ink4/Arf tumor suppression pathway. Thus, therapeutic regimens that increase the dosage of Chd5 in cancer cells will be beneficial for cancer patients with an activating Bmi1 mutatio, by counteracting the inactivating effect of the Bmi1 mutation on the Ink4/Arf tumor suppression pathway. Likewise, cancer therapy targeting Bmi1 also will benefit patients with Chd5 deficiency.

One important aspect of our discovery is that heterozygous deficiency of Chd5 (e.g., the deletion of one allele of the Chd5 gene in the genome) is sufficient to predispose a cell to cancer. This discovery provides a basis for methods of cancer treatment, wherein the endogenous undeleted Chd5 gene in cancer cells is activated to increase the Chd5 protein level in the cells. There are a number of approaches to activating the Chd5 gene. For example, one can target the transcription regulatory region of the gene to promote expression. Alternatively, one can demethylate the gene by using histone deacetylase (HDAC) inhibitors (see, e.g., Sanda et al., Leukemia (2007); kato et al., Clin Cancer Res. 13(15):4538-46 (2007); Qian et al., Mol Cancer Ther. 5(8):2086-95 2006)).

Furthermore, we have demonstrated that excessive Chd5 causes a defect in stem cell renewal and causes stem cells to differentiate, rather than maintain pluripotency. We have also demonstrated that reduced Chd5 dosage promotes stem cell renewal. This discovery provides a basis for methods of regulating stem cell renewal and differentiation by varying the Chd5 levels in the cells.

For example, it has been shown that cancer stem cells are involved in tumorigenesis and tumor maintenance. The invention provides methods of treating cancer by increasing Chd5 dosage in cancer stem cells (e.g., by introducing into cancer stem cells exogenous Chd5 protein or nucleic acid molecules encoding the protein, or by activating the expression of any undeleted endogenous Chd5 allele in the cancer stem cells). The cancer stem cells so treated will undergo cell growth inhibition and/or apoptosis. Conversely, by reducing endogenous Chd5 expression in normal stem cells (e.g., by RNAi described herein), one can promote stem cell renewal in a variety of settings, including bone marrow transplantation and tissue rejuvenation.

Chd5 is a member of the chromo domain superfamily, which is characterized by a large number of evolutionarily conserved proteins containing a chromo (chromatin organizing modulator) domain (Thompson et al., Oncogene 22, 1002-1011). The mammalian chromo domain protein superfamily can be subdivided into five families based on the presence of specific protein motifs that, in addition to the chromo domain that is common to all members of this superfamily, endow each of the five families of proteins with unique function (Jones et al., 2000, Bioessays 22, 124-137). For example, the Pc-G family—first identified as the human homolog of Drosophila Polycomb (Pc), a transcriptional repressor of homeotic genes—contains a Pc box; the HP1 family contains a chromoshadow domain; the RBP family contains an ARD domain; the SUV39H family contains a SET domain; and the Chd family is characterized by the presence of SWI/SNF-type helicase/ATPase—and DNA binding domains.

The Chd family can be further subdivided into subfamilies based on the absence (Chd1/2) or the presence (Chd3/4/5) of two Zn-binding PHD (plant homeodomain) fingers, which facilitate transcriptional activation. With a unique combination of chromo-, helicase-, and DNA binding motifs, Chd5 may epigenetically modify chromatin to regulate gene expression networks affecting development, stem cell fate, and cancer.

Identification of Chd5 as a tumor suppressor functionally validates and extends the idea that chromatin remodeling proteins function in cancer. In exploring the mechanism whereby p53 function is regulated, we found that the DNA damage-induced pathways upstream of p53 were apparently intact in cells heterozygous for the Chd5 locus, but that the ability to effectively induce p53 in response to activated oncogenes was severely compromised. We demonstrated that expression of p19^{Arf}, an upstream inducer of p53 that is activated by oncogene-induced pathways (Lowe and Sherr, supra), is severely compromised by Chd5 deficiency. Since p19^{Arf} sequesters Mdm-2, a negative regulator of p53, the net effect of Chd5 deficiency is to compromise p53 function, thereby facilitating tumorigenesis.

p19^{Arf} and p16^{Ink4a} tumor suppressors are encoded by Ink4/Arf, one of the most commonly inactivated loci in human cancer (Esteller et al., 2001, Cancer Res 61, 3225-3229). p16^{Ink4a} facilitates Rb-, whereas p19^{Arf} stimulates p53-mediated tumor suppressive pathways. Activated oncogenes induce expression of both p19^{Arf} and p16^{Ink4a}, thereby protecting from malignancy (Serrano et al., 1997, Cell 88, 593-602). When these genes are inactivated, however, tumorigenesis can ensue. Importantly, the Ink4/Arf locus is transcriptionally silenced by several members of the Pc-G family, including oncogenes such as Bmi-1, Cbx7, Ring1b, and Ezh2 (Gonzalez et al., 2006, Cell Cycle 5, 1382-1384).

We found that expression of p19^{Arf} at both the transcript level and the protein level is compromised in Chd5 deficient cells. Chd5 deficiency overrides the ability of Ras to induce p19^{Arf} protein, whereas transcript levels of p19 are equivalent to that of wild type cells. The inability of Ras to induce expression of p19^{Arf} that is proportional to the extent of p53 deficiency indicates that the Ink4/Arf locus is inaccessible to transcription in Chd5 compromised cells. This idea is further substantiated by the observation that expression of p16^{Ink4a} was also severely diminished by Chd5 deficiency. These findings support a model in which the chromatin remodeling activity of Chd5 is required for appropriate transcriptional activation of the Ink4/Arf locus.

The 1p36 region has been found to be deleted in a number of human cancers (see above). However, deletion of the entire short arm of human chromosome 1 is a common event in a variety of malignancies, perhaps reflecting genomic instability of many late-stage tumors. The Brodeur group analyzed a panel of 737 primary neuroblastomas and 37 neuroblastoma cell lines, and identified a 729 kb deletion that encompassed 30 genes, including Chd5 (White et al., supra). Similar deletions were found in glioblastoma (Law et al., 2005, Cancer Genet Cytogenet 160, 1-14).

Our discovery was the first to show that Chd5 is a tumor suppressor gene in the 1p36 region and that Chd5 deficiency has a causative role in the tumorigenic process. Furthermore, we discovered that Chd5 deficiency cooperates with activated Ras. We found that heterozygosity of Chd5 predisposes to malignancy and that the wild type locus appears to be retained in both immortalized cells as well as in spontaneous tumors. Thus, complete inactivation of Chd5 by mutation is not a prerequisite for tumorigenesis.

Identification of Chd5 as a tumor suppressor is the first demonstration, to our knowledge, that a member of the Chd family of chromatin remodeling proteins functions in tumorigenesis. We found that Chd5 deficiency inhibits the expression of p16^{ink4a}/p19^{Arf}, showing that Chd5 functions as a transcriptional activator of p16^{Ink4a}/p19^{Arf}. Chd5 may remodel chromatin to favor a transcriptionally active state, facilitating expression of p16^{Ink4a}/p19^{Arf}, thus providing tumor suppression. The SWI/SNF-helicase motif of Chd5 may play a role in the protein's transcriptional activator function.

We found that Chd5 deficiency compromises tumor suppression mediated by both the p19^{Arf}/p53 pathway and the p16^{Ink4a}/Rb pathway. Thus, Chd5 deficiency predisposes to malignancy by crippling tumor suppressive pathways involving p16^{Ink4a}, p19^{Arf}, and p53, and provides a common underlying mechanism for tumorigenesis in diverse types of human cancer.

A human Chd5 polypeptide can be found in the NCBI database with an accession number of Q8TDI0. It has the following sequence:

(SEQ ID NO: 1)

| | |
|---|---|
| MRGPVGTEEE LPRLFAEEME NEDEMSEEED GGLEAFDDFF | 50 |
| PVEPVSLPKK | |
| KKPKKLKENK CKGKRKKKEG SNDELSENEE DLEEKSESEG | 100 |
| SDYSPNKKKK | |
| KKLKDKKEKK AKRKKKDEDE DDNDDGCLKE PKSSGQLMAE | 150 |
| WGLDDVDYLF | |
| SEEDYHTLTN YKAFSQFLRP LIAKKNPKIP MSKMMTVLGA | 200 |
| KWREFSANNP | |
| FKGSSAAAAA AAVAAAVETV TISPPLAVSP PQVPQPVPIR | 250 |
| KAKTKEGKGP | |
| GVRKKIKGSK DGKKKGKGKK TAGLKFRFGG ISNKRKKGSS | 300 |
| SEEDEREESD | |
| FDSASIHSAS VRSECSAALG KKSKRRRKKK RIDDGDGYET | 350 |
| DHQDYCEVCQ | |
| QGGEIILCDT CPRAYHLVCL DPELEKAPEG KWSCPHCEKE | 400 |
| GIQWEPKDDD | |
| DEEEEGGCEE EEDDHMEFCR VCKDGGELLC CDACPSSYHL | 450 |
| HCLNPPLPEI | |
| PNGEWLCPRC TCPPLKGKVQ RILHWRWTEP PAPFMVGLPG | 500 |
| PDVEPSLPPP | |
| KPLEGIPERE FFVKWAGLSY WHCSWVKELQ LELYHTVMYR | 550 |
| NYQRKNDMDE | |
| PPPFDYGSGD EDGKSEKRKN KDPLYAKMEE RFYRYGIKPE | 600 |
| WMMIHRILNH | |
| SFDKKGDVHY LIKWKDLPYD QCTWEIDDID IPYYDNLKQA | 650 |
| YWGHRELMLG | |
| EDTRLPKRLL KKGKKLRDDK QEKPPDTPIV DPTVKFDKQP | 700 |
| WYIDSTGGTL | |
| HPYQLEGLNW LRFSWAQGTD TILADEMGLG KTVQTIVFLY | 750 |
| SLYKEGHSKG | |
| PYLVSAPLST IINWEREFEM WAPDFYVVTY TGDKESRSVI | 800 |
| RENEFSFEDN | |
| AIRSGKKVFR MKKEVQIKFH VLLTSYELIT IDQAILGSIE | 850 |
| WACLVVDEAH | |
| RLKNNQSKFF RVLNSYKIDY KLLLTGTPLQ NNLEELFHLL | 900 |
| NFLTPERFNN | |
| LEGFLEEFAD ISKEDQIKKL HDLLGPHMLR RLKADVFKNM | 950 |
| PAKTELIVRV | |
| ELSQMQKKYY KFILTRNFEA LNSKGGGNQV SLLNIMMDLK | 1000 |
| KCCNHPYLFP | |
| VAAVEAPVLP NGSYDGSSLV KSSGKLMLLQ KMLKKLRDEG | 1050 |
| HRVLIFSQMT | |
| KMLDLLEDFL EYEGYKYERI DGGITGGLRQ EAIDRFNAPG | 1100 |
| AQQFCFLLST | |
| RAGGLGINLA TADTVIIYDS DWNPHNDIQA FSRAHRIGQN | 1150 |
| KKVMIYRFVT | |
| RASVEERITQ VAKRKMMLTH LVVRPGLGSK SGSMTKQELD | 1200 |
| DILKFGTEEL | |
| FKDDVEGMMS QGQRPVTPIP DVQSSKGGNL AASAKKKHGS | 1250 |
| TPPGDNKDVE | |
| DSSVIHYDDA AISKLLDRNQ DATDDTELQN MNEYLSSFKV | 1300 |
| AQYVVREEDG | |
| VEEVEREIIK QEENVDPDYW EKLLRHHYEQ QQEDLARNLG | 1350 |
| KGKRIRKQVN | |
| YNDASQEDQE WQDELSDNQS EYSIGSEDED EDFEERPEGQ | 1400 |
| SGRRQSRRQL | |
| KSDRDKPLPP LLARVGGNIE VLGFNARQRK AFLNAIMRWG | 1450 |
| MPPQDAFNSH | |
| WLVRDLRGKS EKEFRAYVSL FMRHLCEPGA DGAETFADGV | 1500 |
| PREGLSRQHV | |
| LTRIGVMSLV RKKVQEFEHV NGKYSTPDLI PEGPEGKKSG | 1550 |
| EVISSDPNTP | |
| VPASPAHLLP APLGLPDKME AQLGYMDEKD PGAQKPRQPL | 1600 |
| EVQALPAALD | |
| RVESEDKHES PASKERAREE RPEETEKAPP SPEQLPREEV | 1650 |
| LPEKEKILDK | |
| LELSLIHSRG DSSELRPDDT KAEEKEPIET QQNGDKEEDD | 1700 |
| EGKKEDKKGK | |
| FKFMFNIADG GFTELHTLWQ NEERAAVSSG KIYDIWHRRH | 1750 |
| DYWLLAGIVT | |
| HGYARWQDIQ NDPRYMILNE PFKSEVHKGN YLEMKNKFLA | 1800 |
| RRFKLLEQAL | |
| VIEEQLRRAA YLNMTQDPNH PAMALNARLA EVECLAESHQ | 1850 |
| HLSKESLAGN | |
| KPANAVLHKV LNQLEELLSD MKADVTRLPS MLSRIPPVAA | 1900 |
| RLQMSERSIL | |
| SRLTNRAGDP TIQQGAFGSS QMYSNNFGPN FRGPGPGGIV | 1950 |
| NYNQMPLGPY | |
| VTDI | 1954 |

Other human Chd5 polypeptide sequences include, without limitation, NCBI Nos. CAI19894, CAI19890, CAI19891, CAI19883, CAI19450, CAI19451, CAI19449, AAL98962, NP_{—}056372, and AAK56405. An exemplary human Chd5 cDNA is NCBI No. NM_{—}015557. Mouse Chd5 polypeptide sequences include, without limitation, NCBI Nos. AAH98195, XP_{—}993432, XP_{—}914180, and XP_{—}907525. A mouse Chd5 cDNA can be found in the NCBI database with the accession number M5C1079M20. All NCBI sequences mentioned above are incorporated herein by reference in their entirety.

Allelic variations, such as polymorphisms as manifested as single nucleotide polymorphisms (SNPs), occur frequently in eukaryotic genomes. Additionally, small deletions and insertions, rather than SNPs, are not uncommon in the general population, and often do not alter the function of the protein. Accordingly, the above list of Chd5 sequences is illustrative and not exhaustive. Further, sequence variations can be nonnaturally occurring, i.e., can result from human intervention, as by random or directed mutagenesis.

The present invention provides methods of inhibiting abnormal cell growth using a Chd5 protein or an agonist thereof. “Abnormal cell growth” refers to growth that is at an abnormally fast rate, e.g., growth of malignant cancer cells and benign tumor cells. Thus, the methods are useful for treating cancer (including herein hyperproliferation, hyperplasia, neoplasm, and hypertrophy) in a human or non-human subject (e.g., mouse, rat, dog, and primate). The goal of the treatment is preventing tumorigenesis, preventing tumor progression (including metastasis), preventing angiogenesis at tumor sites, inducing tumor regression, and/or preventing cancer recurrence. Cancers that can be treated by the methods of this invention include epithelial-originated cancers (e.g., skin cancer such as melanoma, thyroid cancer, breast cancer, colorectal cancer, cervical cancer, ovarian cancer, and prostate cancer), neural-associated cancers (e.g., neuroblastoma, oligodendroglioma, glioma, meningioma, and pheochromocytoma), mesenchymal-associated cancers (e.g., kibernoma), sarcomas, and lymphoid malignancies (e.g., leukemia, and lymphomas). Typically, the cancers that are treated by these methods are characterized by Chd5 deficiency—with heterozygous or homozygous deletion of the Chd5 gene, a mutation in the Chd5 gene that reduces the activity of Chd5, an abnormality in certain components of the Chd5-mediated cell cycle regulatory pathway which renders the Chd5 activity deficient, or epigenetic silencing.

Prior to the treatment, it may be preferable to confirm that the patient has a Chd5 deficiency. The goal will be more customized treatment. Those with Chd5 deficiency will be treated more aggressively with the Chd5 protein or an agonist thereof, and those who do not display Chd5 deficiency may avoid unnecessary and potentially toxic treatments. One way to detect Chd5 gene deletion (which can be detected as 1p36 deletion or D4Mit190-D4Mit51 deletion) is genomic profiling as done by microarray analysis. One such method is Representational Oligonucleotide Microarray Analysis (ROMA), a genome-wide scanning method capable of identifying copy number alterations in cells at high resolution (Lucito et al., 2003, Genome Res. 13:2291-2305; Sebat et al., 2004, Science 305:525-528). One can also use, e.g., the GENECHIP arrays, the SNP arrays, and the EXON arrays available from Affymetrix to perform microarray analysis of genomic profiles for detecting Chd5 deletion. Other methods for detecting Chd5 deletion includes polymerase chain reaction (PCR), Southern blotting, and dot blotting, using primers and/or probes from the Chd5 gene or its surrounding region. The primers and probes may hybridize to the entirety or part (e.g., at 20, 25, 35, or 40 contiguous nucleotides) of the Chd5 gene, including allelic variants of the gene, or its flanking sequences. Preferably, the primers and probes hybridize to a relatively unique part of the gene so as to reduce background noise signal. Genomic DNA from a normal individual or from a healthy tissue of the cancer patient can be used as a control for detecting deletion. In some embodiments, the patient is selected for Chd5 treatment because his cancer cells show a compromised p53 or Rb tumor suppression function; Chd5 treatment may help restore or bypass the p53 and Rb deficiency.

The Chd5 protein used in the cancer treatment of this invention (which can be introduced into a patient, by, e.g., protein infusion methods) can be the full length Chd5 protein, such as those illustrative above, or a protein containing one or more functional domains of the full length protein. Functional domains of Chd5 include, without limitation, the CHDNT domain (e.g., amino acid residues 146-200 of SEQ ID NO:1), the PHD domains (e.g., residues 345-390 and residues 418-463 of SEQ ID NO:1), the Chromo domain (e.g., residues 592-644 of SEQ ID NO:1), the SNF2_N domain (e.g., residues 703-999 of SEQ ID NO:1), the helicase C domain (e.g., residues 1059-1138 of SEQ ID NO:1), the DUF domains (e.g., residues 1295-1359 and 1372-1531), and the CHDCT2 domain (e.g., residues 1730-1903 of SEQ ID NO:1). The Chd5 protein useful in this invention may contain one or more of the above domains and optionally heterologous domains that enhance the therapeutic function of the protein.

For example, the Chd5 protein of this invention may be a fusion protein comprising one or more Chd5 domains connected to a heterologous polypeptide, i.e., a polypeptide that does not naturally occur in contiguity with the Chd5 fusion partner. The fusion protien can consist entirely of a plurality of fragments of the native Chd5 protein in altered arrangement; in such a case, any of the Chd5 fragments can be considered heterologous to the other Chd5 fragments in the fusion protein.

The heterologous polypeptide included within the fusion protein of the present invention is at least 6 amino acids in length, often at least 8 amino acids in length, and preferably, at least 15, 20, and 25 amino acids in length. The heterologous sequences can target the Chd5 polypeptide to a selected cell by binding to a cell surface receptor, for example, a receptor that is preferentially expressed on a target cell (e.g., a cancer cell or a stem cell), prolong the serum life of the Chd5 polypeptide (e.g., an IgG Fc region), make the Chd5 polypeptide detectable (e.g., a luciferase or a green fluorescent protein), facilitate purification (e.g., His tag, FLAG, etc.), facilitate secretion of recombinantly expressed proteins (e.g., into the periplasmic space or extracellular milieu for prokaryotic hosts, into the culture medium for eukaryotic cells, through incorporation of secretion signals and/or leader sequences). Other useful protein fusions of the present invention include fusions that permit, e.g., the nuclear localization of the Chd5 polypeptide.

The proteins and protein fragments of the present invention can also usefully be fused to protein toxins, such as Pseudomonas exotoxin A, diphtheria toxin, shiga toxin A, anthrax toxin lethal factor, ricin, or other biologically deleterious moieties in order to effect specific ablation of cells that bind or take up the proteins of the present invention.

The Chd5 proteins used in this invention can be composed of natural amino acids linked by native peptide bonds, or can contain any or all of nonnatural amino acid analogues, nonnative bonds, and post-synthetic (post-translational) modifications, either throughout the length of the polypeptide or localized to one or more portions thereof. However, the range of such nonnatural analogues, nonnative inter-residue bonds, or post-synthesis modifications will be limited to those that do not interfere with the biological function of the polypeptide.

Techniques for incorporating non-natural amino acids during solid phase chemical synthesis or by recombinant methods are well established in the art. For instance, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., 1995, Biochem. Biophys. Res. Corn. 209:817-821), and various halogenated phenylalanine derivatives.

The isolated Chd5 polypeptides can also include non-native inter-residue bonds, including bonds that lead to circular and branched forms. The isolated Chd5 polypeptides can also include post-translational and post-synthetic modifications, either throughout the length of the protein or localized to one or more portions thereof. For example, when produced by recombinant expression in eukaryotic cells, the isolated polypeptide can include N-linked and/or O-linked glycosylation, the pattern of which will reflect both the availability of glycosylation sites on the protein sequence and the identity of the host cell. Further modification of glycosylation pattern can be performed enzymatically. As another example, the Chd5 proteins may also include an initial modified methionine residue, in some cases resulting from host-mediated processes.

Amino acid analogues having detectable labels are also usefully incorporated during synthesis to provide a labeled polypeptide (e.g., biotin, various chromophores, or fluorophores). The Chd5 proteins can also usefully be conjugated to polyethylene glycol (PEG). PEGylation increases the serum half life of proteins administered intravenously.

Production of the Chd5 proteins used in this invention can optionally be followed by purification from the producing cells. Producing cells include, without limitation, recombinant cells overexpressing the proteins, naturally occurring cells (e.g., cancer cells) overxpression the polypeptides, or established cancer cell lines overexpressing the proteins. If purification tags have been fused through use of an expression vector that appends such tags, purification can be effected, at least in part, by means appropriate to the tags, such as use of immobilized metal affinity chromatography for polyhistidine tags. Other techniques common in the art include ammonium sulfate fractionation, immuno-precipitation, fast protein liquid chromatography (FPLC), high performance liquid chromatography (HPLC), and preparative gel electrophoresis. Purification of chemically-synthesized peptides can readily be effected, e.g., by HPLC.

The Chd5 proteins can also be administered in form of an expression vector comprising a coding sequence of the protein operably linked to transcription regulatory sequences that allow the expression of the protein in the host cells. The expression vector can be a viral vector based upon a replication incompetent lentivirus, retrovirus, adenovirus, or adeno-associated virus (AAV). Such viruses may preferentially target fast-dividing cells such as cancer cells.

The transcription regulatory sequences on the expression vectors include promoters and enhancer elements, those that facilitate RNA processing, such as transcription termination, splicing signals and/or polyadenylation signals, and those that facilitate translation, such as ribosomal consensus sequences. Other transcription control sequences include operators, silencers, and the like. Use of such expression control elements, including those that confer constitutive or inducible expression, and developmental—or tissue-regulated expression are well-known in the art. Constitutively active promoters include, without limitation, a CMV promoter, EF1α, retroviral LTRs, and SV40 early region. Inducible promoters include, without limitation, a tetracycline-inducible promoter, a metallothionine promoter, the IPTG/lacI promoter system, the ecdysone promoter system, and the “lox stop lox” system for irreversibly deleting inhibitory sequences for translation or transcription. Tissue-specific promoters that can be used in driving expression of Chd5 include, without limitation, the keratin 5 promoter, the keratin 14 promoter, the p63 promoter, the whey acidic protein promoter, the Em promoter, the GFAP promoter, and the nestin promoter.

This invention also provides cells and nonhuman animals comprising a genome containing an inactivating Chd5 mutation (e.g., heterozygous or homozygous deletion or inactivating disruption) that renders the cells deficient in Chd5. These cells and animals can be made by standard techniques such as homologous recombination. The inactivating disruption can be conditional; that is, the targeted disruption of the Chd5 gene is effected at a controlled manner, e.g., inducibly, at certain stages of an animal's development, and/or in certain tissues. This can be achieved by, e.g., placing a cre-lox-cre cassette in the Chd5 gene and co-introducing into the cell a Cre recombinase gene under the control of a promoter that is inducible, developmental-specific, and/or tissue-specific (see above). The knockout can also be achieved by using small interfering RNA molecules (siRNAs) against Chd5 transcripts. The expression of the siRNAs can be similarly controlled. In some embodiments, the cells and animals of the invention additionally comprise an expression construct comprising an activated oncogene such as H-ras, Akt, Bcl-2, myc, and Bmi-1.

The animals of the invention can be a transgenic animal or a chimeric animal. A “transgenic animal” is a non-human animal, preferably a mammal, more preferably a cow, goat, sheep, or rodent such as a rat or mouse, all of whose cells contain the engineered genetic elements discussed above. A chimeric animal is an non-human animal only some of whose cells carry the engineered genetic elements, and those cells can be introduced into the animal in an embryonic stage by, e.g., microinjection (blastocyst injection), or in a later stage by, e.g., implantation into a target organ.

The cells and animals of this invention can be used to study the effect of the Chd5 gene on tumorigenicity and tumor development, to study the role of Chd5 on normal tissue development and differentiation, to identify via array CGH regions of the genome whose amplification and deletion is correlated with Chd5 status. The cells and animals can be used to determine the efficacy of a candidate compound in preventing or treating cancer, or otherwise inducing apoptosis and senescence. In one such method, one can administer to the animal a candidate compound and observe the effect of the compound on tumor development, maintenance, angiogenesis and/or progression in the animal. Regression and/or reduction of tumor size in the presence of the compound is indicative of the effectiveness of the compound. Similarly, the effect of a candidate compound on the level of Chd5 mRNA, protein, or activity in the animal or cell lines derived thereof (or cell lines transfected with the gene) can be used to identify the candidate as an agonist or antagonist.

The animal model can also be used to identify other components in the Chd5-mediated cell cycle regulatory pathway. To do this, a detailed expression profile of gene expression in tumors undergoing regression or regrowth due to the inactivation or activation of the Chd5 gene is established. Techniques used to establish the profile include the use of suppression subtraction (in cell culture), differential display, proteomic analysis, serial analysis of gene expression (SAGE), and expression/transcription profiling using cDNA and/or oligonucleotide microarrays. Then, comparisons of expression profiles at different stages of cancer development can be performed to identify genes whose expression patterns are altered.

This invention further provides methods for identifying a Chd5 agonist. Such an agonist is useful to treat cancer patients, or otherwise treat patients with a condition that benefits from increased apoptosis and senescence of certain cells/tissues. An agonist is identified by its ability to increase Chd5 protein level or activity level in a cell. Cells and animals of this invention can be used for these methods. Chd5 protein levels can be determined by standard methods such as Western blotting, immunohistochemistry, and ELISA. Chd5 protein levels also correlate with, and thus can be indirectly determined by, the expression and activity levels of p53 and p53-regulated genes such as p21 and Mdm-2. Chd5 protein levels can also be indicated by Chd5 transcript levels, which in turn can be determined by e.g., Northern blotting, and real time quantitative PCR.

The Chd5 proteins or agonists can be administered to a patient in a pharmaceutically accepted carrier. Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). Physiologically acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

Pharmaceutical formulation is a well-established art, and is further described in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X), the disclosures of which are incorporated herein by reference in their entireties. Conventional methods, known to those of ordinary skill in the art of medicine, can be used to administer the pharmaceutical formulation(s) to the patient.

Typically, the pharmaceutical formulation will be administered to the patient by applying to the skin of the patient a transdermal patch containing the pharmaceutical formulation, and leaving the patch in contact with the patient's skin (generally for 1 to 5 hours per patch). Other transdermal routes of administration (e.g., through use of a topically applied cream, ointment, or the like) can be used by applying conventional techniques. The pharmaceutical formulation(s) can also be administered via other conventional routes (e.g., parenteral, subcutaneous, intrapulmonary, transmucosal, intraperitoneal, intrauterine, sublingual, intrathecal, or intramuscular routes) by using standard methods. In addition, the pharmaceutical formulations can be administered to the patient via injectable depot routes of administration such as by using 1-, 3-, or 6-month depot injectable or biodegradable materials and methods.

Regardless of the route of administration, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose. The effectiveness of the method of treatment can be assessed by monitoring the patient for known signs or symptoms of a disorder.

The pharmaceutical compositions of the invention may be included in a container, package or dispenser alone or as part of a kit with labels and instructions for administration. These compositions can also be used in combination with other cancer therapies involving, e.g., radiation, photosensitizing compounds, anti-neoplastic agents and immunotoxics.

The following examples are intended to illustrate the methods and materials of the present invention. Suitable modifications and adaptations of the described conditions and parameters normally encountered in the art that are obvious to those skilled in the art are within the spirit and scope of the present invention.

### Example 1

### Generation of Mouse Strains with Rearrangements Corresponding to Human 1p36

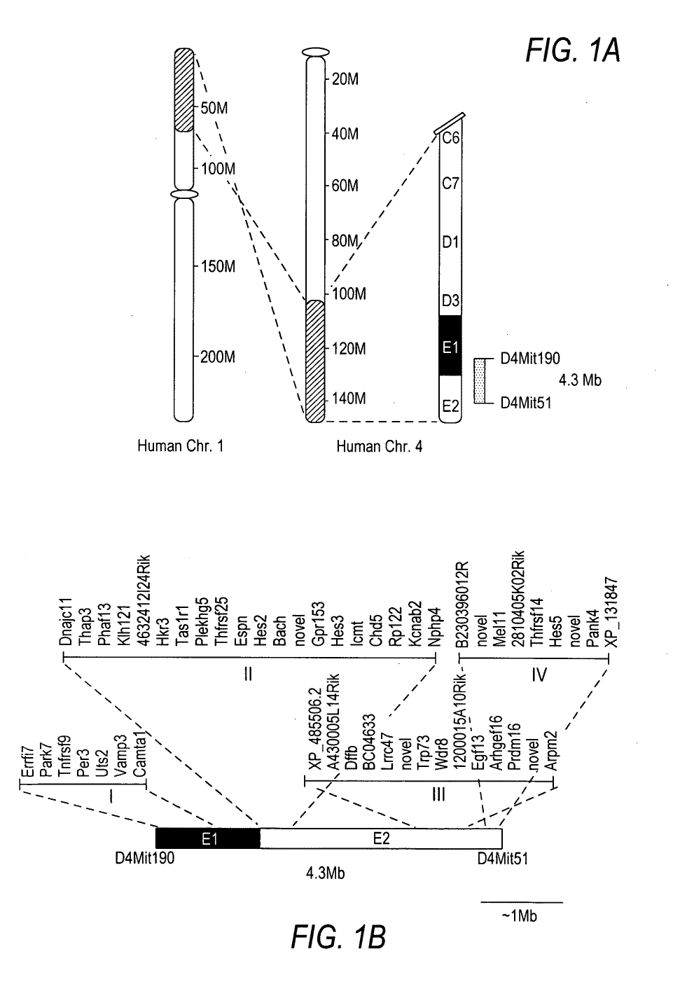

To functionally identify novel tumor suppressor genes mapping to human 1p36, mouse strains with defined rearrangements in the corresponding region of the mouse genome were generated using chromosome engineering (FIG. 1A) (Ramirez-Solis et al., 1995, Nature 378, 720-724) reviewed in (Mills, et al., supra). Genes mapping to human 1p36 lie within a conserved linkage group that maps to the distal portion of mouse chromosome 4. Therefore, mouse strains with chromosome engineered deletions (also called deficiencies, df) or duplications (dp) of this region provide genetic models for functionally characterizing genes mapping to human 1p36. Mouse strains harboring df and dp alleles have decreased—and increased dosage of genes mapping within the rearranged interval, respectively.

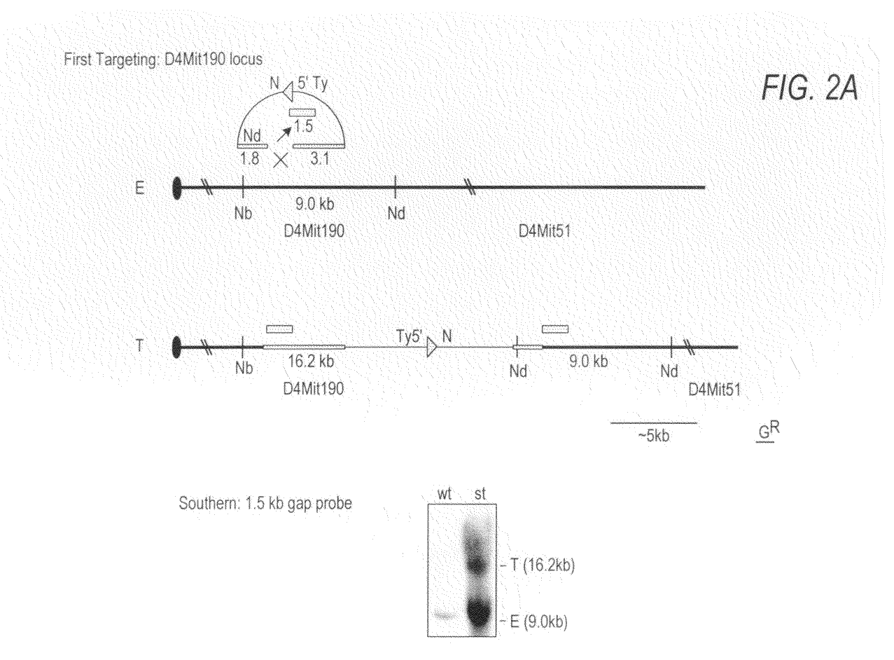

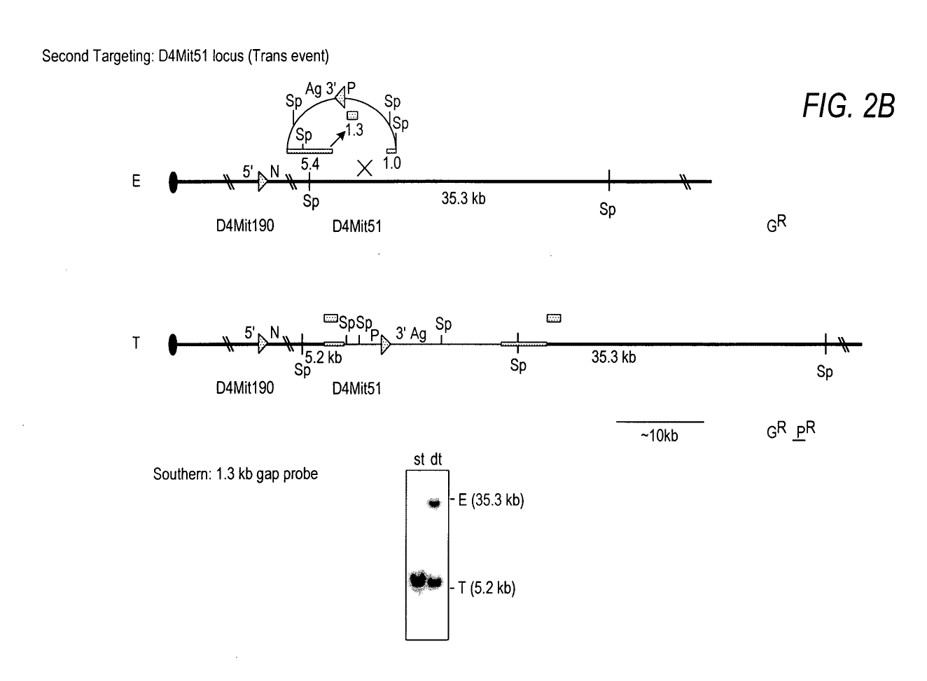

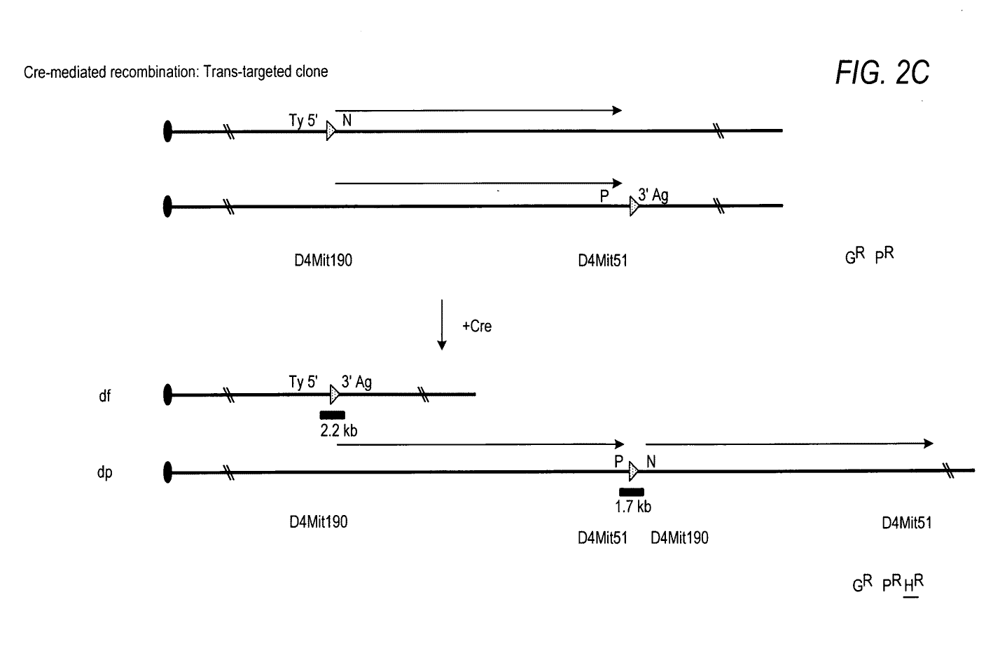

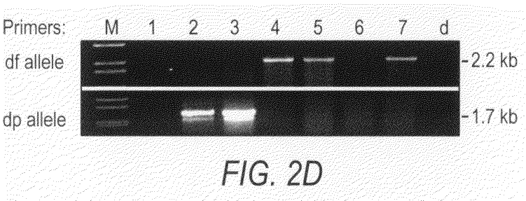

The D4Mit190-D4Mit51 region contains the genes shown (FIG. 1B; Table 1). To generate df and dp alleles of this interval, gene targeting was used to sequentially modify the selected endpoints in embryonic stem (ES) cells (FIG. 2). Insertion-type targeting vectors specific for D4Mit190 and D4Mit51 were isolated from a resource of genomic libraries of essentially pre-made gene targeting insertion vectors contained within bacteriophage lambda backbones (Adams et al., 2004, Nat Genet 36, 867-871; Zheng et al., 1999, Nucleic Acids Res 27, 2354-2360). After converting the lambda vectors to plasmids by infecting the Cre-expressing bacterial strain with the isolated bacteriophage, the genomic inserts were sequenced to determine the orientation of the insert contained within the plasmid backbone relative to the loxP sites. Each construct was converted to an insertion targeting vector by linearization within the region of homology by removing the gap region (FIG. 2A, B). The gap fragment provides a probe for identifying homologous recombinants since this region is present if and only if accurate gap repair dependent homologous recombination at the target locus has occurred (Hasty et al., 1991, Mol Cell Biol 11, 4509-4517). Gene targeting at D4Mit190 was performed, and G418-resistant clones were screened by Southern analysis using the gap probe to identify homologous recombinants, which occurred with an efficiency of 30.7% (FIG. 2A).

TABLE 1
Genes mapping to D4Mit190-D4Mit51

| | Accession | Accession | |
|---|---|---|---|
| Gene | Number (human) | Number (mouse) | Gene Ontology |
| Errfi1¹ | NM_018948 | NM_133753 | Protein Binding |
| Park7 | BC008188 | NM_020569 | RNA binding, protein folding, |
| | | | dopamine uptake, adult locomotory |
| | | | behavior |
| Tnfrs9 | NM_001561 | NM_011612 | Receptor activity, membrane |
| | | | protein, induction of apoptosis, |
| | | | immune response |
| Per3¹ | NM_016831 | NM_011067 | Signal transduction, circadian |
| | | | rhythm |
| Uts2 | NM_006786 | NM_011910 | Hormone activity, extracellular |
| | | | space |
| Vamp3 | NM_004781 | NM_009498 | Protein assembly complex, vesicle |
| | | | docking during exocytosis, |
| | | | membrane fusion, synaptosome |
| Camta1^{1,2} | NM_015215 | XM_355539 | Calmodulin binding, Wnt signaling |
| | | | pathway |
| 2310058O09Rik | — | AK144150 | — |
| 9230110K08Rik | — | AK020331 | Regulation of transcription, DNA |
| | | | dependent |
| Dnajc11^{1,2} | NM_018198 | NM_172704 | Protein folding, heat shock protein |
| | | | binding |
| Thap3 | NM_138350 | NM_175152 | Nucleic acid binding, metal ion |
| | | | binding |
| Phf13 | NM_153812 | NM_172705 | Regulation of transcription, DNA |
| | | | and protein binding |
| Klhl21 | — | NM_001033352 | Protein binding |
| Hkr3¹ | NM_005341 | NM_133879 | Regulation of transcription, DNA |
| | | | and protein binding, metal ion |
| | | | binding |
| Tas1r1 | NM_138697 | NM_031867 | GPCR protein signaling pathway, |
| | | | taste receptor activity |
| Nol9 | NM_024654 | NM_028727 | Carboxypeptidase activity, sugar |
| | | | binding and hydrolase activity |
| Plekhgh5 | — | NM_001004156 | Regulation of Rho protein signal |
| | | | transduction |
| Tnfrsf25 | NM_020631 | NM_033042 | Receptor activity, membrane |
| | | | protein, induction of apoptosis, |
| | | | immune response |
| Espn | — | NM_207687 | Actin filament network formation |
| Hes2¹ | NM_019089 | NM_008236 | Regulation of transcription, DNA |
| | | | dependent |
| Acot7 | NM_007274 | NM_133348 | Serine esterase activity, fatty acid |
| | | | catabolism |
| Gpr153 | NM_207370 | NM_178406 | Rhodopsin like receptor activity, |
| | | | signal transduction |
| Hes3 | NM_001024598 | NM_008237 | Regulation of transcription, DNA |
| | | | dependent |
| D330010C22Rik | NM_173795 | NM_001033489 | Ubiquitin protein ligase activity |
| Icmt | NM_012405 | NM_133788 | C-terminal carboxy methyl |
| | | | transferase, protein modification |
| Rpl22 | — | NM_009079 | Structural constituent of ribosome, |
| | | | RNA binding, protein biosynthesis |
| Chd5^{1,2} | NM_015557 | XM_988338 | Regulation of transcription, DNA |
| | | | dependent, chromatin assembly and |
| | | | disassembly, ATP dependent |
| | | | helicase activity, chromatin |
| | | | modification, metal ion binding |
| Kcnab2^{1,2} | NM_003636 | NM_010598 | Voltage gated potassium channel |
| | | | activity |
| Nphp4 | NM_015102 | BC044732 | Visual behavior, protein binding, |
| | | | signal transduction |
| Gm833 | — | NM_001033414 | — |
| XP_485506 | NM_018836 | XM_001000455 | — |
| BC049688 | — | AK131708 | — |
| A430005L14Rik | NM_207356 | NM_175287 | — |
| Dffb¹ | NM_001004285 | NM_007859 | DNA binding, nicotinate |
| | | | phosphoribosyl transferase activity, |
| | | | nuclease activity, DNA |
| | | | fragmentation during apoptosis |
| Lrrc47 | NM_020710 | NM_201226 | — |
| Ccdc27 | NM_152492 | NM_001033455 | — |
| Trp73^{1,2} | NM_005427 | NM_011642 | Regulation of transcription, DNA |
| | | | dependent, mismatch repair, DNA |
| | | | damage response, induction of |
| | | | apoptosis |
| Wdr8 | NM_017818 | NM_021499 | — |
| 1200015A19Rik | NM_182752 | NM_026388 | Identical protein binding |
| Megf6 | NM_001409 | XM_001004037 | Calcium ion binding, structural |
| | | | molecule activity |
| Arhgef16 | NM_014448 | XM_149562 | |
| Prdm16¹ | NM_022114 | NM_027504 | Regulation of transcription, DNA |
| | | | dependent, Nucleic acid binding, |
| | | | metal ion binding |
| 5930403L14Rik | — | NM_001033398 | — |
| Actrt2 | NM_080431 | NM_028513 | Structural component of |
| | | | cytoskeleton, protein binding |
| B230396O12Rik | — | NM_172878 | Binding |
| Mmel1 | NM_033467 | NM_013783 | Neprilysin activity, Golgi apparatus, |
| | | | protease activity, metalopeptidase |
| | | | activity, zinc ion binding |
| 2810405K02Rik | NM_152371 | NM_025582 | — |
| Tnfrsf14 | NM_003820 | NM_178931 | Receptor activity, membrane |
| | | | protein, induction of apoptosis, |
| | | | immune response |
| Hes5 | NM_001010926 | NM_010419 | Regulation of transcription, DNA |
| | | | dependent |
| Pank4 | NM_018216 | NM_172990 | Pantothenate kinase activity, ATP |
| | | | binding, coenzyme A biosynthesis |
| Plcl4 | XM_933992 | NM_175556 | Phosphoinositide phospholipase C |
| | | | activity, signal transducer, lipid |
| | | | catabolism |
| Pex10/ | NM_002617 | XM_131847 | Peroxisome organization and |
| XP_131847.5 | | | biogenesis, protein binding, protein |
| | | | import to peroxisome matrix |

¹Short hairpins generated
²Short hairpins validated

After determining that the D4Mit190-targeted clones were germline competent by performing blastocyst injection and demonstrating that the modified allele was transmitted and generated phenotypically normal progeny, singly-targeted clones were targeted a second time using the D4Mit51 targeting vector and accurate targeting of puromycin-resistant clones was assessed by Southern analysis using the D4Mit51 gap probe; homologous recombination occurred in 36.1% of the G418-resistant clones (FIG. 2B). Targeting at D4Mit190 occurred with a frequency of 30.7%; germline competent clones were targeted at D4Mit51 with an efficiency of 36.1%. Eleven independently generated doubly targeted clones were expanded and electroporated with a Cre-expressing construct to facilitate recombination between the integrated loxP sites, and clones were grown in media containing hypoxanthine aminopterin thymidine (HAT) to select for rearrangements (FIG. 2C). HAT-resistant clones were expanded and subjected to sib drug selection to determine the type of rearrangement that had occurred. This drug selection assay revealed that 4 clones were dfl+ and 7 were dfldp, indicating that targeting had occurred in Cis and Trans orientation, respectively (data not shown; FIG. 2C) (Mills, et al., supra).

Two independently generated dfldp clones (which were genetically balanced since they did not have any net gain or loss of genes within the rearranged interval) were used to generate chimeric mice by blastocyst injection. Male chimeras were mated to wild type females and progeny were genotyped at weaning by PCR to identify dfl+ and dp/+ mice. Whereas dfl+ mice (n=41) were generated out of 8 litters, dp/+ weanling mice were not obtained, even though the dp/+ allele should have been transmitted to half of the Agouti progeny. When embryos from similar crosses were harvested during late gestation, however, dfl+ (n=22) and dp/+ (n=20) progeny were obtained (FIG. 2D). These observations indicate that deficiency of the D4Mit190-D4Mit51 interval is tolerated during embryonic development, but that duplication of this region causes perinatal lethality.

### Example 2

### dp/+ mefs have Decreased Proliferation and Enhanced Senescence

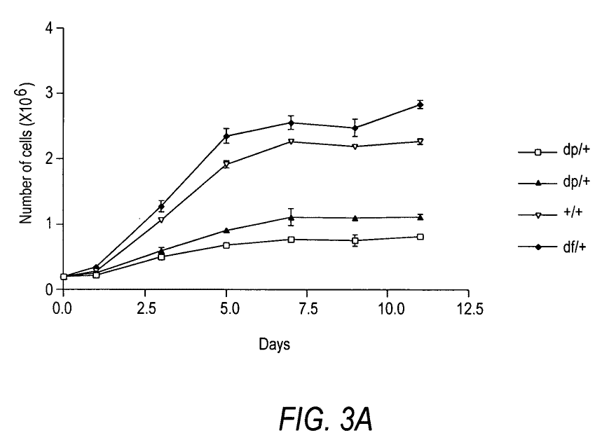

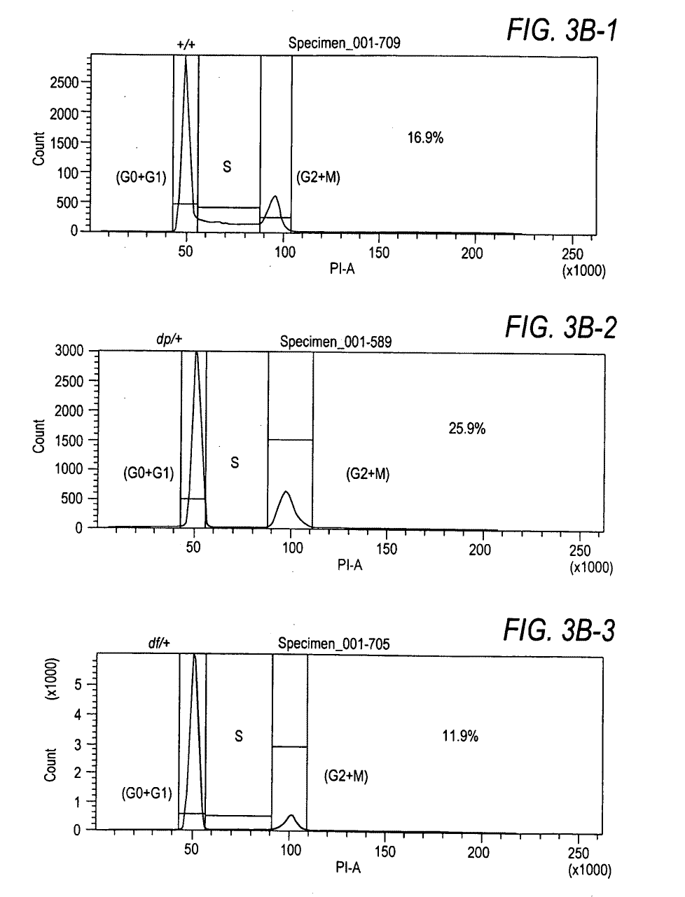

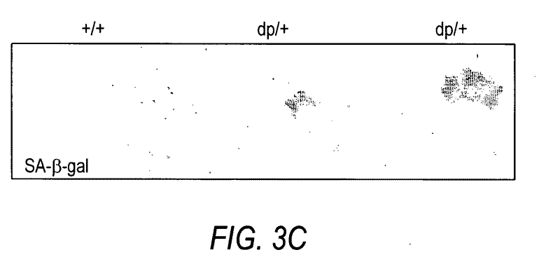

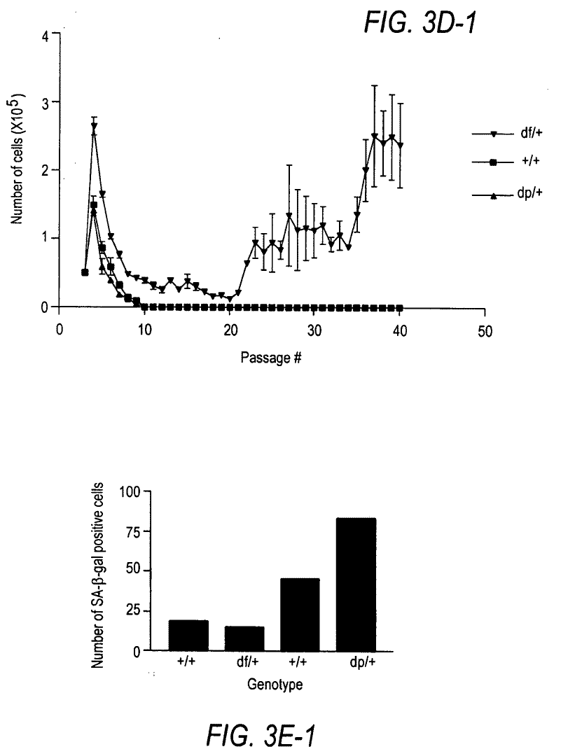

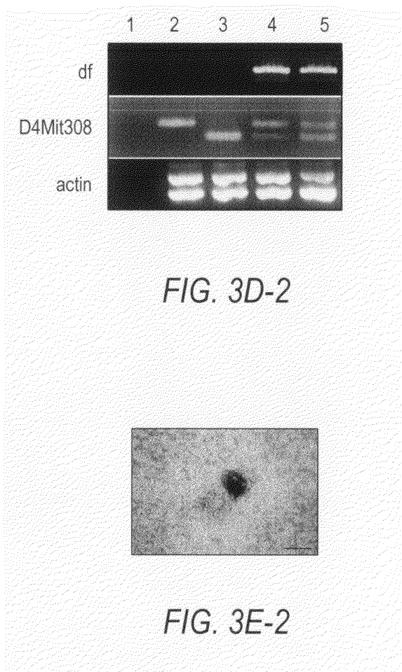

To evaluate the cellular phenotype dp/+ mice, we harvested mouse embryonic fibroblasts (mefs) from embryonic day 13.5 (E13.5) embryos obtained from +/+ females that had been crossed to dfldp chimeras. Mefs were subjected to a cell proliferation assay in which equivalent numbers of cells were plated and cells were harvested and counted at 2-day intervals (FIG. 3A). While +/+ mefs proliferated robustly, dp/+ mefs had reduced proliferative potential. To investigate the proliferative defect of dp/+ mefs further, flow cytometry was used to determine the population of cells in different stages of the cell cycle (FIG. 3B). While 16.9% of the +/+ mef population was in G2/M, 25.9% of the dp/+ mef population was in G2/M. Consistent with the inability of dp/+ mefs to progress through G2/M, many of the dp/+ cells appeared senescent by morphological criteria: they were flattened, enlarged and extended multiple processes. To determine whether these cells were senescent, +/+ and dp/+ mefs were assayed for senescence associated beta galactosidase (SA-β-gal) activity, a marker of senescent cells (FIG. 3C) (Dimri et al., 1995, Proc Natl Acad Sci USA 92, 9363-9367). Whereas very few senescent cells were detected in early passage +/+ or dfl+ mef cultures, a significant increase in the number of senescent cells was found in dp/+ cultures. These analyses indicate that duplication of the D4Mit190-D4Mit51 region dramatically compromises cellular proliferation and enhances cellular senescence.

### Example 3

### dfl+ mefs have Enhanced Proliferation, Immortalize and Form foci Spontaneously

In contrast to the decreased proliferation of dp/+ mefs, dfl+ mefs had a significantly enhanced proliferative potential compared to +/+ cells (FIG. 3A). In addition, flow cytometry indicated that while 16.9% of the +/+ mef population was in G2/M, only 11.9% of the dfl+ population was in this phase of the cell cycle (FIG. 3B). To examine whether dfl+ mefs have an extended cellular lifespan, we used the 3T3 protocol in which an equivalent number of mefs from mice of different genotypes were initially plated, and cells were counted and replated at 3-day intervals (FIG. 3D, upper panel). Wild type—and dp/+ mefs could not be maintained for more than 8-10 passages, a finding consistent with previous reports. In contrast to the limited cellular lifespan of +/+ and dp/+ mefs, dfl+ mefs could be serially passaged extensively (i.e., 48 times to date) indicating that immortalized cells are easily selected for in the dfl+ culture.

We next asked whether loss of the wild type allele accompanies immortalization, we performed PCR analysis using simple sequence length polymorphism (SSLP) primers that would discriminate between the wild type and the df alleles, which are from the C57BL/6 and129S5 mouse strains, respectively (FIG. 3D, lower panel). Both the wild type and the df allele were detectable in early passage mefs obtained from F1 dfl+ embryos, as expected. PCR analysis of immortalized dfl+ cells (passage 37) indicated that the wild type chromosome was retained.

To assess whether mefs heterozygous for the D4Mit190-D4Mit51 interval undergo contact inhibition, cells were grown at high density to evaluate their ability to form foci (FIG. 3E). Whereas +/+ cells ceased to proliferate upon contact and formed monolayers dfl+ cells frequently formed foci with a frequency of approximately 10-5. These findings indicate that heterozygosity of the D4Mit190-D4Mit51 interval leads to enhanced proliferation, sensitivity to immortalization and spontaneous foci formation.

### Example 4

### Deletion of the D4Mit190-D4Mit51 Interval Causes Hyperproliferation, Whereas Duplication of this Region Causes Apoptosis In Vivo

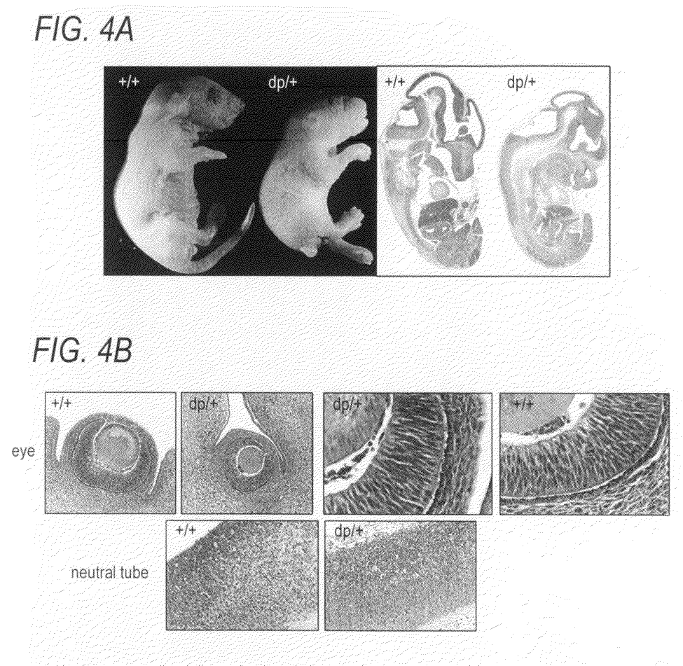

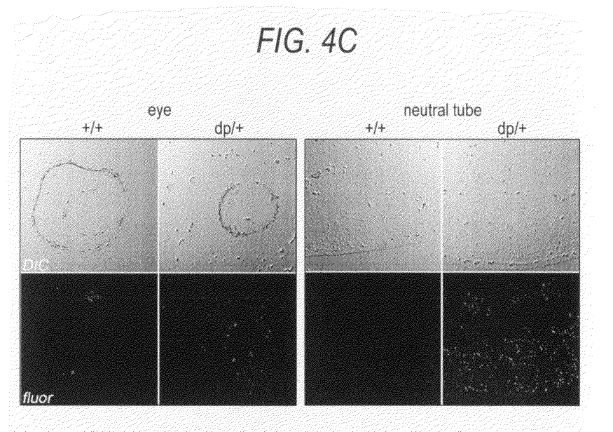

To determine whether the increased or decreased proliferation of dfl+ and dfl+ cells in culture has a consequence in vivo, the phenotype of embryos from +/+ females that had been mated to dfldp chimeras was analyzed. dfl+ mice were grossly normal and indistinguishable from +/+ progeny at E17.5. However, dfl+ adult mice frequently developed hyperplasia in a variety of tissues. In contrast to the normal phenoytpe of dfl+ embryos, late gestation dp/+ embryos had striking developmental abnormalities (exencephaly, eye defects [microphthalmia], a conspicuous curling of the tail) that caused them to die perinatally (FIG. 4A). Histological analysis revealed that the neural tube contained a large number of cells that appeared to be undergoing apoptosis (FIG. 4B). To determine if these cells were in fact apoptotic, we performed TUNEL assays on sagittal sections of control and dp/+ embryos (FIG. 4C). Whereas very few TUNEL-positive cells were detected in +/+ embryos, there was a marked increase in the number of apoptotic cells in tissues of dp/+ embryos, which was particularly notable in the neural tube. This analysis indicates that hyperproliferation and apoptosis are caused by deletion and duplication of the D4Mit190-D4Mit51 interval, respectively, providing in vivo evidence that this region is a potent regulator of cellular proliferation and survival.

### Example 5

### The dp/+ Phenotype is Caused by Enhanced Gene Dosage

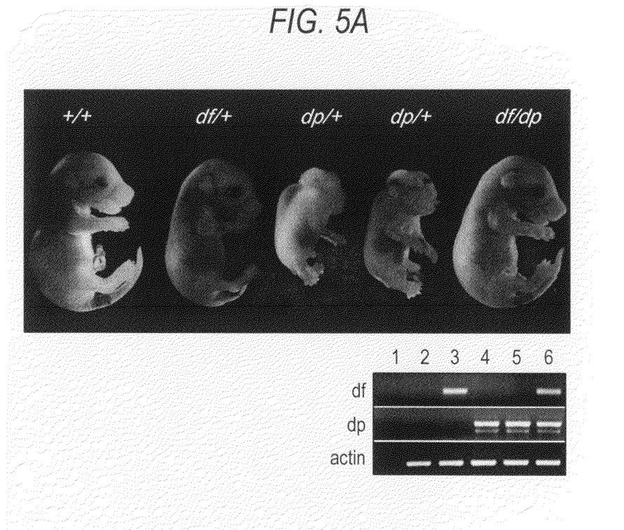

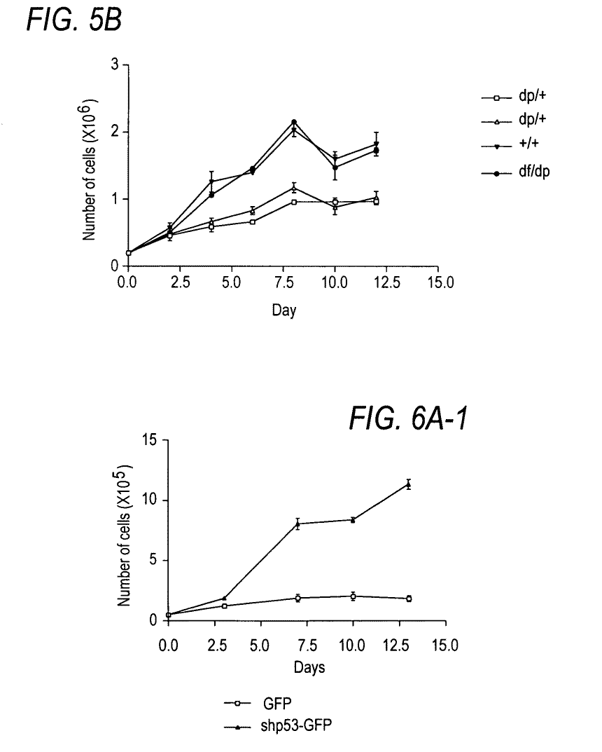

dp/+ embryos had severe anomalies and died perinatally due to excessive apoptosis, whereas dfl+ embryos appeared phenotypically normal at this stage and were generated in a Mendelian ratio. We hypothesized that the excessive apoptosis of dfl+ mice was caused by enhanced dosage of the D4Mit190-D4Mit51 interval. An alternative possibility was that the phenotype of dp/+ mice was due to altered gene function resulting from the large-scale chromosome rearrangement that had been generated. To test between these two possibilities, we evaluated the in vivo consequence of reducing the 3N dosage of dp/+ mice to 2N by analyzing the phenotype of dfldp embryos (FIG. 5). Chimeric mice harboring both df and dp D4Mit190-D4Mit51 alleles were mated to dfl+ mice, and progeny were harvested at E17.5. Wild type and dfl+ embryos appeared phenotypically normal whereas dp/+ embryos had striking developmental defects, as shown above (FIG. 5A; see FIG. 4A). dfldf embryos were not obtained in these crosses, suggesting that D4Mit190-51 homozygosity is embryonic lethal. Importantly, dfldp embryos were indistinguishable from wild type embryos, demonstrating that the df allele functionally rescued the developmental defects characteristic of dp/+ embryos. Furthermore, dfldp mice (n=11) obtained from dfldp intercrosses were viable and fertile, whereas weanling dp/+ mice were not obtained when dfldp chimeras were crossed to +/+ mice (see above).

To determine whether the ability of the df allele to rescue the developmental defects of dp/+ mice was cell intrinsic, mefs were generated from these mice and were subjected to the proliferation assay described above (FIG. 5B). While two independent lines of dp/+ mefs had reduced proliferation comparable to that shown in FIG. 3A, proliferation of dfldp mefs was equivalent to that of wild type mefs. These analyses provide direct genetic evidence that both the enhanced apoptosis of dp/+ mice in vivo and the reduced proliferation of dp/+ mefs in culture are due to increased dosage of the D4Mit190-D4Mit51 interval. Therefore, proliferation, apoptosis, and senescence are tightly regulated by dosage of this region.

### Example 6

### The dp/+ Phenotype is p53-Dependent

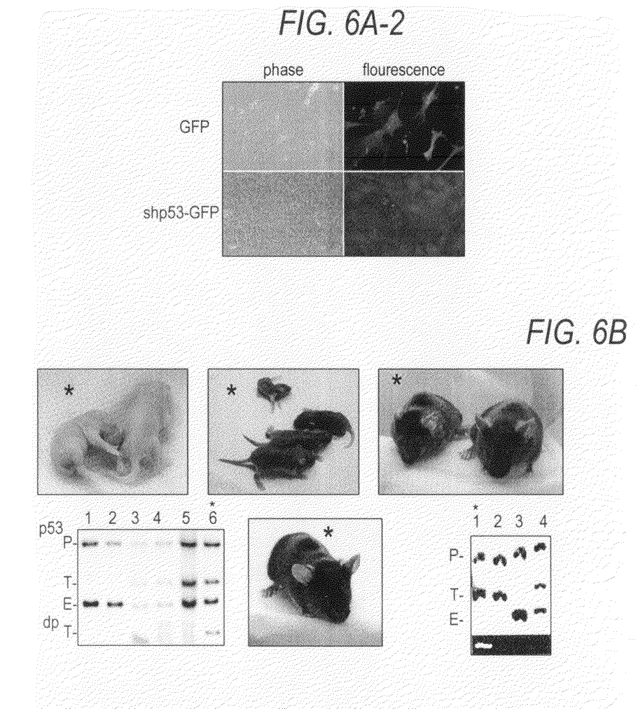

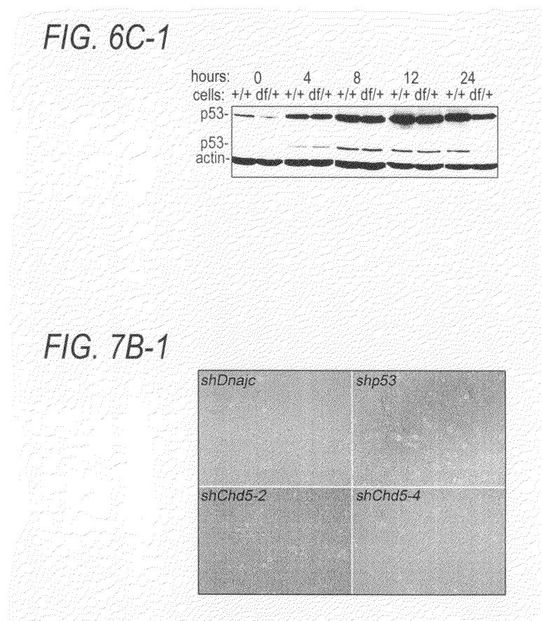

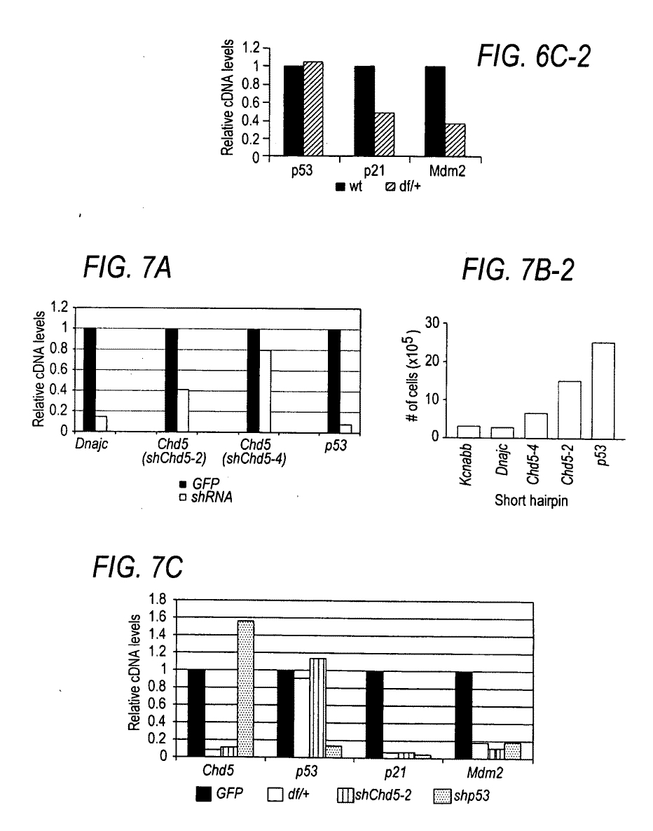

Given that both apoptosis and cellular senescence were observed in dp/+ cells and that both of these tumor suppressive mechanisms can be modulated by p53, we next examined whether the compromised proliferation of dp/+ cells was p53-dependent. Cultures of dp/+ mefs were infected with retrovirus expressing either GFP—or a short hairpin RNA (shRNA) specific for p53 (FIG. 6A, left). Control (GFP-infected) dp/+ cultures had very low levels of proliferation and infected cells frequently displayed a characteristic senescent morphology as seen above (see FIG. 3A, C). In contrast, dp/+ cultures infected with retrovirus expressing a p53 shRNA proliferated robustly, indicating that p53 knockdown provided a significant growth advantage in dp/+ cells. Proliferation assays of GFP—and shp53-infected mefs revealed that the proliferative defect was bypassed by p53 knockdown (FIG. 6A, right). This analysis indicates that the compromised proliferation caused by increased dosage of D4Mit190-D4Mit51 is p53 dependent in cultured cells.

We next asked whether p53 deficiency, which bypassed the senescence of dp/+ mefs, would also protect from apoptosis in vivo and rescue the perinatal lethality. dfldp and p53+/− mice were intercrossed and the phenotype of the progeny analyzed (FIG. 6B). When litters (dp/+; p53+/−) were analyzed within one day after birth, viable newborns with developmental defects affecting the tail and eye were noticed (FIG. 6B, upper left). However, these defects were significantly less severe than those of dp/+ mice (compare FIG. 6B to 4A, 5A), suggesting that p53 heterozygosity partially rescues the developmental defects of dp/+ mice. Indeed, when these mice were genotyped, they proved to be dp/+; p53+/− (FIG. 6B, bottom left). Although many of these mice became runted, developed ataxia, and did not survive until weaning, some dp/+; p53+/− mice survived to adulthood (FIG. 6B, upper middle, right). In addition, subsequent crosses between dfldp; p53+/− and p53+/− mice produced viable dp/+; p53−/− progeny (FIG. 6B, lower middle), demonstrating that absence of p53 bypassed the lethality of dp/+ mice in vivo. These analyses provide direct genetic evidence that both the proliferative—and apoptotic defects of dp/+ cells are rescued by p53 deficiency, indicating that increased dosage of the D4Mit190-D4Mit51 interval modulates proliferation, senescence, and apoptosis in a p53-dependent manner.

### Example 7

### p53 is Compromised in dfl+ Cells

An important advantage of the chromosome engineering strategy is that the phenotypic consequence of the gain and loss of a precise genomic region can be directly compared and contrasted. Since increased dosage of D4Mit190-D4Mit51 was found to enhance p53 function, we reasoned that reduced dosage of this region would lead to the opposite phenotype, i.e., Aug. 16, 2006 a compromise in p53 function. To examine whether p53 levels were altered in dfl+ mefs, p53 expression was assessed by real-time PCR (FIG. 6C, left). However, we found that p53 expression at the transcript level was essentially equivalent in +/+ and dfl+ mefs. To explore whether p53 was expressed similarly in +/+ and dfl+ cells at the protein level, p53 expression was assessed by Western analysis (FIG. 6C, right). p53 protein levels were lower in dfl+ mefs, indicating that basal p53 expression is compromised in cells heterozygous for the df allele.

To determine whether the lower p53 levels in dfl+ cells compromised p53 function, real-time PCR was used to assess expression of the p53 target genes p21 and Mdm-2 as readout for p53 transcriptional activity (FIG. 6C, left). Indeed, p21 and Mdm-2 expression was dramatically reduced in dfl+ mefs. To investigate whether DNA damage could induce p53 similarly in +/+ and dfl+ cells, mefs were treated with adriamycin to induce p53 protein and p53 levels were assessed by Western analysis (FIG. 6C, right). Whereas we had shown that basal levels of p53 were compromised in dfl+ mefs, DNA damage triggered a robust induction of p53 protein in both +/+ and dfl+ cells, indicating that dfl+ cells can induce p53 in response to DNA damage as efficiently as wild type cells. However, whereas p53 levels were maintained in +/+ mefs 24 hours after adriamycin treatment, p53 levels began to decline in dfl+ cells 24 hours following DNA damage. These analyses indicate that deficiency of the D4Mit190-D4Mit51 interval compromises p53 function.

To extend these findings, we analyzed df/+, +/+, and dp/+ mefs for p53 expression by immunofluorescence, and found a direct correlation between dosage and p53 expression. These analyses revealed that p53 expression was compromised and enhanced in df/+ and dp/+ cells, respectively, further demonstrating that D4Mit190-51 positively regulates p53.

### Example 8

### Identification of the Proliferation-Suppressing Gene Mapping to D4Mit190-D4Mit51

The D4Mit190-D4Mit51 interval includes 52 annotated genes (see FIG. 1B and Table 1, supra). Reasoning that increased dosage of a single gene within this region modulates proliferation, apoptosis, and senescence, we screened for genes that would functionally rescue the compromised proliferation phenotype of dp/+ cells when depleted. Proof of principal experiments for this screen were that the proliferation defect of dp/+ mefs could be rescued by the df allele as well as by shRNA-mediated knockdown of p53 (see FIGS. 5B, 6A), indicating that reducing dosage restores proliferation and that RNAi-mediated knockdown using retroviral vectors is effective in dp/+ mefs.

To prioritize between candidate genes, we assessed the Gene Ontology (GO) terms of genes mapping to the D4Mit190-D4Mit51 region, and selected a number of potential candidate genes (see Table 1). Retroviral constructs encoding shRNAs that also express GFP were generated against Camtal, Chd5, Dffb, Dnajc11a, Errfi1, Hes2, Hkr3a, Kcnabb, p73, Per3, Prdm16. These constructs were tested for their ability to knockdown their specific targets in +/+ cells by real-time PCR (e.g., FIG. 7A). Next, a number of these hairpins were scored for their ability to bypass the proliferation defect of dp/+ cells (e.g., FIG. 7B). dp/+ mefs infected with control retrovirus encoding GFP proliferated poorly (e.g., FIG. 6A). We found that cultures infected with shRNA constructs specific for Kcnabb, Camtal, and Dnajc proliferated poorly and were indistinguishable from those infected with the GFP control (e.g., FIG. 7B). Unexpectedly, dp/+ mefs expressing hairpins specific for the p53 homolog p73 proliferated even more poorly than dp/+-GFP controls, indicating that p73 deficiency exacerbated rather than rescued the dp/+ phenotype. Thus, Camta1, Dnajc, Kcnabb, and p73 were not the genes responsible for the proliferative defect of cells with enhanced dosage of the D4Mit190-D4Mit51 region.

In contrast, two distinct shRNA constructs specific for the chromodomain helicase DNA-binding protein Chd5 efficiently rescued the proliferative defect of dp/+ mefs (FIG. 7B). Importantly, the extent of knockdown correlated well with the efficiency of rescue, indicating that the level of Chd5 tightly regulates proliferation. The most efficient hairpin against Chd5, shChd5-2, restored proliferation of dp/+ cells nearly as well as did p53 knockdown, and occurred later than the rescue achieved by p53 knockdown, suggesting that p53 is genetically downstream of Chd5. This finding is consistent with the ability of p53 deficiency to rescue the senescent—and apoptotic phenotype of dp/+ cells in culture and in vivo, respectively (see FIG. 6A, B).

The ability of Chd5 knockdown to restore proliferation in dp/+ cells indicates that enhanced dosage of Chd5 is responsible for suppressing proliferation of dp/+ mefs. To further demonstrate that Chd5 negatively regulates proliferation and is not simply a feature of dp/+ mefs, we tested whether knockdown of Chd5 could enhance proliferation in wild type cells. +/+ mefs were infected with retrovirus expressing shRNAs specific for Dnajc11a or Chd5 and the effect on proliferation assayed. As was the case with dp/+ mefs, +/+ cells in which Dnajc11a was knocked down proliferated similarly to GFP-infected cells. In contrast, Chd5 knockdown markedly enhanced proliferation of +/+ mefs, as was also the case when p53 was knocked down in +/+ cells. These experiments indicate that Chd5 knockdown enhances proliferation in +/+ mefs. Together with the ability of Chd5 knockdown to bypass the proliferative defect in dp/+ mefs, these results indicate that Chd5 is the gene within the D4Mit190-D4Mit51 interval that encodes a protein that negatively regulates proliferation and induces potent tumor suppressive mechanisms.

### Example 9

### Chd5 Positively Regulates p53-Mediated Pathways

We had demonstrated that Chd5 is the gene within the D4Mit190-D4Mit51 interval that encodes a potent negative regulator of proliferation, determined that the phenotype of dp/+ cells is p53 dependent, and discovered that dfl+ cells have impaired p53 function. Therefore, we next examined whether +/+ cells in which Chd5 was specifically depleted using RNAi phenocopy dfl+ cells in their inability to modulate p53 (FIG. 7C). Expression of Chd5 was reduced in both dfl+ mefs as well as in +/+ cells in which Chd5 was knocked down. +/+-GFP, dfl+, +/+-shChd5-2, and +/+-shp53 mefs were analyzed for p53 expression by real-time PCR (FIG. 7C). As was the case for dfl+ cells, +/+-shChd5 mefs expressed p53 transcript at levels that were essentially equivalent to that of +/+-GFP cells, whereas RNAi-mediated knockdown of p53 depleted p53 expression, as expected. We next analyzed the expression of the p53 target genes p21, Mdm-2, PUMA, Bax and PML as readout for p53 transcriptional activity using real-time PCR (e.g., FIG. 7C). Whereas +/+-GFP mefs expressed these target genes robustly, expression was significantly reduced in both dfl+ and +/+-shChd5-2 mefs, indicating that p53 transcriptional activity is compromised in Chd5 deficient cells. Importantly, p53 transcriptional activity in mefs in which Chd5 was knocked down paralleled that of dfl+ cells, indicating that Chd5 is the gene within the D4Mit190-D4Mit51 interval responsible for modulating p53. These analyses demonstrate that p53 levels are compromised when Chd5 is depleted, indicating that Chd5 positively regulates p53-mediated pathways.

### Example 10

### Chd5 Suppresses Oncogenic Transformation

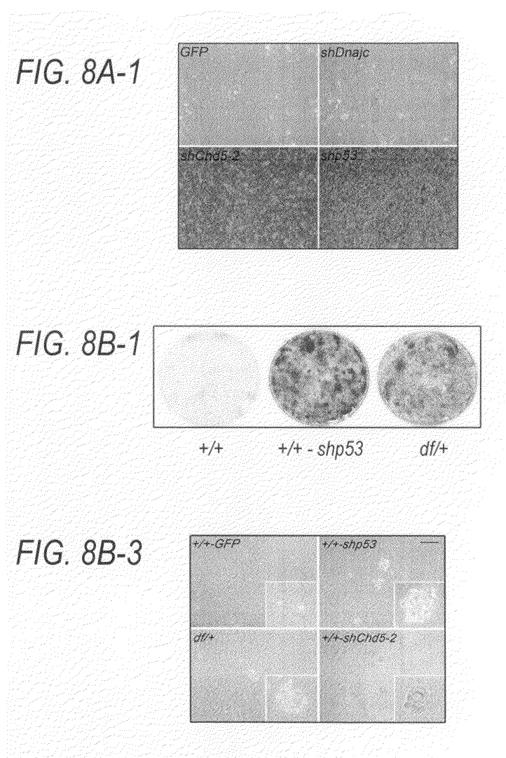

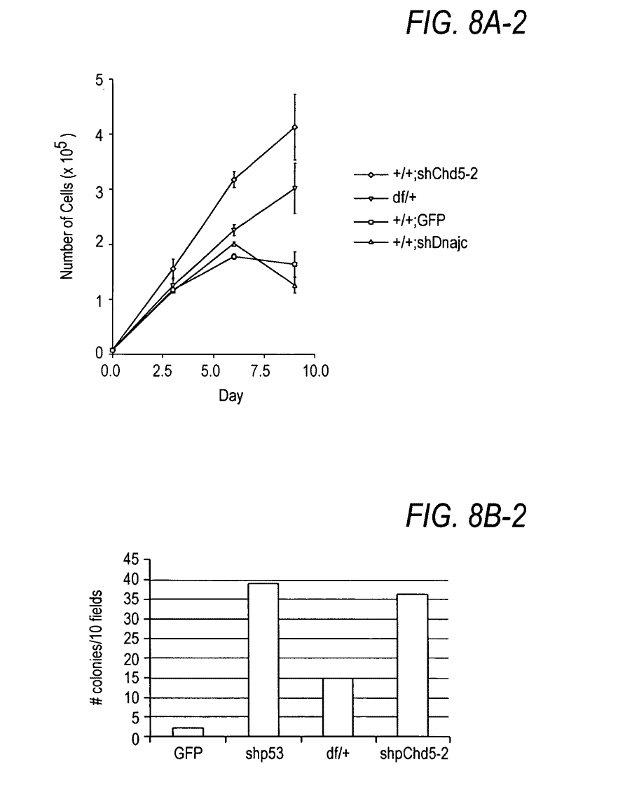

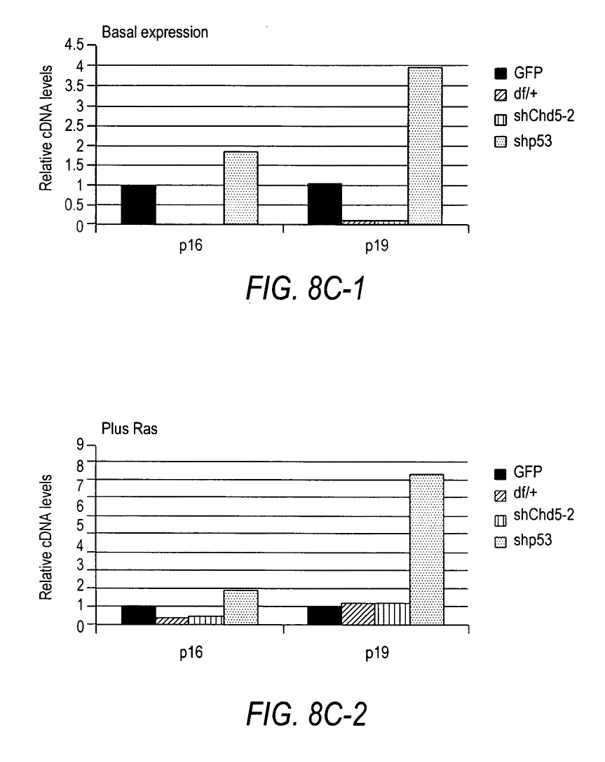

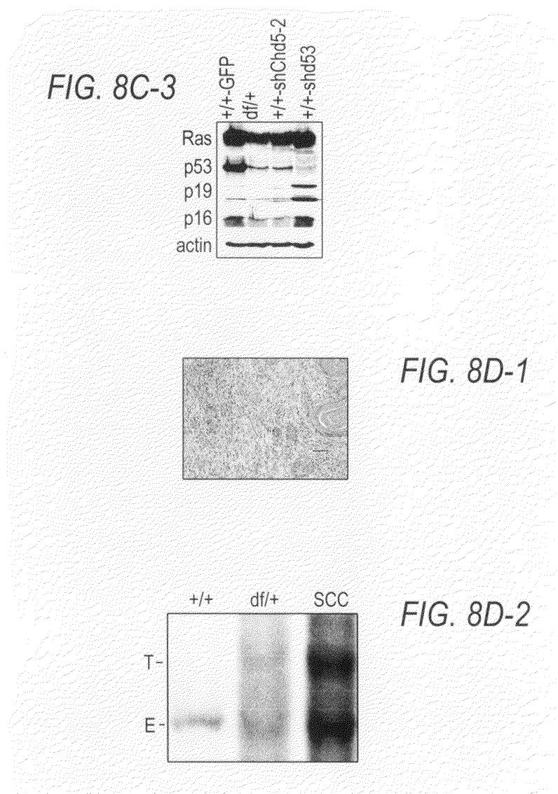

Since p53 deficient cells are susceptible to oncogenic transformation and deficiency of either the D4Mit190-D4Mit51 interval or Chd5 alone compromises p53 function, we assessed whether oncogenic Ras could transform these cells. First, +/+-GFP, +/+-shDnajc, +/+-shp53, dfl+, and +/+-shChd5-2 cells were infected with retrovirus expressing RasV12 and the proliferation phenotype analyzed (e.g., FIG. 8A). In agreement with previous reports, +/+-GFP mefs expressing oncogenic Ras became senescent and proliferation was severely limited, whereas +/+ cells in which p53 had been knocked down proliferated robustly in response to oncogenic Ras. Importantly, Ras induced robust proliferation in both dfl+ mefs as well as in +/+ mefs in which Chd5 had been knocked down. Interestingly, RNAi-mediated knockdown appeared to induce proliferation more effectively than engineered heterozygosity of the df allele in the presence of Ras (FIG. 8A, lower panel).

To determine whether dfl+ cells could be transformed by activated Ras, colony assays were performed in Ras-expressing—+/+-GFP, dfl+, and +/+-shp53 mefs (FIG. 8B, upper panels). Indeed, dfl+ cells were readily transformed by oncogenic Ras and were comparable in their colony forming ability to p53 deficient cells, whereas Ras was not able to transform +/+-GFP cells. We next assessed whether specific knockdown of Chd5 in—+/+ cells would phenocopy dfl+ cells in their sensitivity to transformation by infecting dfl+ and +/+-shChd5-2 cells with Ras and assessing anchorage independent growth in soft agar (FIG. 8B, middle—and lower panels). +/+-GFP mefs expressing oncogenic Ras were incapable of forming foci in this assay, whereas +/+ cells in which p53 had been knocked down were transformed by Ras and formed foci. We found that both Ras-expressing—dfl+ and +/+-shChd5-2 mefs grew robustly in soft agar and readily formed foci, indicating that these cells were prone to oncogenic transformation. These analyses indicate that both heterozygous deficiency of the entire D4Mit190-D4Mit51 interval as well as specific knockdown of Chd5 sensitizes cells to oncogenic transformation, demonstrating that Chd5 functions as a tumor suppressor, and protects against malignant transformation.

### Example 11

### Chd5 Modulates p53 Via Regulation of p16^{Ink4a}/p19^{Arf}

We next investigated the mechanism whereby Chd5 regulates p53 and suppresses Ras-mediated transformation, by determining whether dfl+ and Chd5 knockdown cells were impaired in their ability to modulate pathways upstream of p53. Our previous results indicated that DNA damage-induced pathways that regulate p53 were apparently intact, but that oncogene-induced pathways that regulate p53 were perturbed by either heterozygosity of the D4Mit190-D4Mit51 interval or by deficiency of Chd5 (see FIGS. 6C and 7C). Therefore, we focused on identifying perturbations in the oncogene-induced pathways that involve the Ink4/Arf locus encoding the cyclin dependent kinase inhibitor p16^{Ink4a} and p19^{Arf}.

To determine whether p16^{Ink4a} and p19^{Arf} function were compromised in dfl+ and Chd5 knockdown cells, we used real-time PCR to quantitate expression of p16^{Ink4a} and p19^{Arf} at the transcript level in cells prior to and following expression of oncogenic Ras (FIG. 8C, upper, middle panels). p16^{Ink4a} and p19^{Arf} were induced at the transcript level in p53 deficient cells and Ras enhanced this effect. In contrast, expression of both p16^{Ink4a} and p19^{Arf} were significantly compromised in both dfl+ and Chd5 knockdown cells, both prior to and following Ras expression. Although p19^{Arf} expression was induced to levels equivalent to that wild type cells at the transcript level, p16^{Ink4a} expression remained compromised in Chd5 deficient cells (FIG. 8C, middle panel).

To determine whether p16^{Ink4a} and p19^{Arf} protein levels were compromised in cells heterozygous for D4Mit190-D4Mit51 or in cells in which Chd5 had been knocked down, we performed Western analyses using p16^{Ink4a}—and p19^{Arf}-specific antibodies in Ras-expressing dfl+ and +/+-shChd5-2 cells (FIG. 8C, lower panel). Ras induced expression of both p16^{Ink4a} and p19^{Arf} in +/+-GFP cells, and expression of p19^{Arf} in response to Ras was even more robust in mefs in which p53 had been knocked down, as reported previously (Lowe et al., 2003, Curr Opin Genet Dev 13, 77-83). In contrast, expression of p19^{Arf} in response to Ras was severely diminished in dfl+ mefs. Importantly, specific knockdown of Chd5 resulted in a compromise in Ras-mediated induction of p19^{Arf} that closely paralleled that of dfl+ cells, indicating that Chd5 is the gene within the D4Mit190-D4Mit51 region that encodes a protein responsible for inducing the Ink4/Arf locus in response to oncogenic stimulation. Ras-mediated induction of p16^{Ink4a} was also substantially reduced at the protein level in response to Ras in both cells heterozygous for the D4Mit190-D4Mit51 deletion and in cells rendered deficient for Chd5 by RNAi-mediated knockdown.

To extend these findings, we analyzed df/+, +/+, and dp/+ mefs for p19^{Arf} and p16^{Ink4a} expression by immunofluorescence. This analysis revealed that expression of both p19^{Arf} and p16^{Ink4a} was compromised and enhanced in df/+ and dp/+ cells, respectively supporting the finding that Chd5 positively regulates the Ink4/Arf locus.

To functionally validate the hypothesis that Chd5 modulates proliferation by facilitating p19^{Arf}, we used RNAi to deplete p19^{Arf} in dp/+ mefs. We found that knockdown of p19^{Arf} bypassed the proliferation defect of dp/+ cells. Proliferation assays revealed that dp/+ mefs proliferated more efficiently than GFP-infected control cells (p=0.005) and achieved proliferation levels comparable to that of Chd5 knockdown (p=0.435). In contrast, knockdown of p16^{Ink4a} proliferated only slightly better than controls (p=0.15). These analyses provide functional evidence that p19^{Arf} is maintained by a gene within the D4Mit190-51 interval.

To extend this study, we obtained dp/+ mice in a p16/p19-deficient background. We showed that heterozygosity of Ink4/Arf deficiency partially rescued the dp/+ phenotype in vivo, further demonstrating the genetic connection between Chd5 and Ink4/Arf Since regulation of the Ink4/Arf locus by members of the Pc-G family of proteins such as Bmi 1 have been shown to have a profound impact on stem cell fate, this study suggests that Chd5 affects stem cell fate.

The above data demonstrate that Chd5 maintains p53 levels by facilitating expression of p19^{Arf} and that deficiency of Chd5 predisposes to malignant transformation by compromising the p19^{Arf} /p53 pathway.

### Example 12

### Chd5 is a Tumor Suppressor Gene

Our findings that increased dosage of Chd5 reduces proliferation and evokes the tumor suppressive mechanisms of cellular senescence and apoptosis, coupled with the fact that decreased dosage of Chd5 enhances proliferation, compromises p19^{Arf}/p53-mediated pathways and leads to immortalization and cellular transformation, strongly suggested that Chd5 might function as a potent tumor suppressor in vivo. Consistent with the findings that both dfl+ cells and mefs in which Chd5 had been knocked down were susceptible to Ras-mediated transformation, our analysis of tumorigenesis in df/+ and control cohorts to date indicated that df/+ mice were also prone to spontaneous tumors (FIG. 8D). Indeed, a df/+ mouse developed spontaneous squamous cell carcinoma of the skin, and another df/+ mouse developed lymphoma at about 10 months of age (FIG. 8D, upper panel); kibernoma (a mesenchymal neoplasm), adenocarcinoma of the breast, and sarcoma of the uterus were also found. Thus, the mouse models showed the same types of tumors characteristic of 1p36 deletion.

Importantly, the finding that mammary tumors occur spontaneously in df/+ mice evidenced that CHD5 acts as a tumor suppressor in breast cancer. This finding shows that the CHD5 mutations identified in human breast cancer (Sjoblom et al., Science 314:268-74 (2006)) are functionally important.

The above data evidenced that the mef phenotypes described here were relevant to tumorigenesis in an in vivo setting. Chd5 was expressed in cells that formed tumors when 1p36 was deleted, as well as in cell types that gave rise to the tumors we observed in df/+ mice.

To examine whether tumors that formed in df/+ mice retained the +/+ allele, genomic DNA was subjected to Southern analysis. This demonstrated that the wild type chromosome was retained in spontaneous tumors that formed in df/+ mice. Together, these analyses further demonstrate that heterozygous deficiency of the D4Mit190-D4Mit51 interval predisposes to malignancy in vivo, and that Chd5 is the gene in this region that functions as a tumor suppressor.

We tested whether Chd5 deficiency predisposed to tumorigenesis in vivo in athymic nude mice. Control mefs (+/+-GFP and +/+-shDnajc) failed to form tumors (0 neoplasms/10 injections), whereas p53 knockdown mefs formed robust tumors (10 neoplasms/10 injections). In contrast, both df/+ cells and +/+ mefs in which Chd5 was specifically knocked down formed tumors readily (20 neoplasms/20 injections and 11 neoplasms/12 injections, respectively). These studies conclusively demonstrate that Chd5 functions as a tumor suppressor.

### Example 13

### CHD5 is Deleted in Human Tumors

After having shown that Chd5 is a tumor suppressor in mice, we tested whether CHD5 functions in human cancer by analyzing expression of genes mapping to chromosome 1p in a panel of 54 human gliomas (Bredel et al., Cancer Research 65:8679-8689 (2005)). CGHPRO data analysis tools (Chen et al., BMC Bioinformatics 6:85 (2005)) and a circular binary segmentation algorithm (Olshen et al., Biostatistics 5:557-572 (2004)) were used to define deletions. This analysis mapped CHD5 to a 5.4-Mb minimal common deletion region (MCR) between 1p36.32 and 1p36.22. The corresponding gene expression portraits (Bredel et al., supra) compared the CHD5 expression levels in normal human brain and in tumors with—or without CHD5 deletion. Whereas non-deleted tumors expressed CHD5 at levels comparable to normal brain (P=0.66, independent t-test), tumors with deletion had a significant decrease in CHD5 expression (P=0.00006). The narrow range of CHD5 expression in deleted tumors contrasted markedly with the much wider range in tumors without deletion.

To assess whether there was a significant association between gene dosage and CHD5 expression in the tumors, we used an integrative strategy that combined a modification of signal-to-noise ratio computation and permutation testing (Juric, Methods Mol Biol, 2007) in deleted-versus morphology matched non-deleted tumors. The measure of statistical significance in terms of the false discovery rate (the q value (Storey and Tibshirani, PNAS 100:9440-9445 (2003)) discloses significance for the genetic level in driving expression levels of CHD5 in the tumors (q <0.05). These findings further demonstrate that Chd5 is a novel tumor suppressor and pinpoint deletion of the 1p36 interval encompassing CHD5 as an initiating event in tumorigenesis.

Thus, Chd5's role as a tumor suppressor is consistent with our finding that CHD5 maps within an MCR in human cancer. Previous studies reported that 1p36 deletions in human cancer that included CHD5 (Bello et al., Int. J. Cancer 64:207-210 (1995); Bieche et al., Cancer Research 53:1990-1994 (1993); Moley et al., Cancer Research 52:770-774 (1992); Praml et al., Oncogene 11:1357-1362 (1995); White et al., PNAS 92:5520-5524 (1995); White et al., Oncogene 24:2684-2694 (2005); Law et al., Cancer Genet Cytogenet 160:1-14 (2005); Thompson et al., Oncogene 22:1002-1011 (2003)). However, none of these studies pinpointed Chd5′s role as a tumor suppressor gene.

### Example 14

### Generation and Characterization of Chd5/CHD5-Specific Antibodies

The data shown above demonstrated that heterozygosity of Chd5 crippled a tumor suppressive network, thereby initiating the tumorigenic process. To determine whether Chd5 protein expression was retained in tumors that developed in Chd5 compromised mice, we developed antibodies specific for Chd5. Two different affinity-purified polyclonal antibodies, Chd5-232 and Chd5-312, which were generated against peptides 232-246 and 312-326 of mouse Chd5, respectively. These regions of the mouse Chd5 protein were selected because they are distinct from other members of the Chd family, and yet they are conserved in the human Chd5 protein (human CHD5). Both of these antibodies performed robustly in western blot analyses (e.g., the brain lysate of mice), and recognized the same molecular weight protein. In addition, Chd5-232 recognized human CHD5 in Western analyses (e.g., the human HCT116 cell line). We also demonstrated that both of these antibodies immunoprecipitated endogenous Chd5 effectively.

Due to their excellent performance in Western and immunoprecipitation assays as well as their ability to detect both mouse Chd5 and human CHD5, these antibodies and like antibodies are useful in studying the tumor suppressive role of these novel proteins. These antibodies are useful in assessing Chd5 expression in tumors that arise spontaneously or in concert with activated oncogenes in mouse models. These antibodies are useful for determining whether Chd5 protein expression is in fact lost in mice homozygous for the disrupted Chd5 allele.

These antibodies are useful in assessing CHD5 expression in human archival tumors and CHD5 status in human cancer, using, e.g., Western and immunohistochemical analyses. For example, we have analyzed the expression of CHD5 at the protein level in a variety of human cell lines, including MCF7 (epithelial-like carcinoma), LN308 (glioblastoma), HeLa (epithelial carcinoma), HCT116 (epithelial carcinoma), A431 (epidermoid carcinoma), U937 (monocytic lymphoma), U118.MG (glioblastoma), T47D (epithelial-like carcinoma), MDA-MB-486 (epithelial-like adenocarcinoma), IMR-90 (fibroblast), 293 (transformed epithelial cells), A204 (epithelial-like rhabdomyosarcoma), A2058 (epithelial-like melanoma), A-549 (epithelial-like carcinoma), RKO (epithelial carcinoma), Sk-N-BE (epithelial-like neuroblastoma), and U2Os (epithelial-like sarcoma).

Furthermore, Chd5-specific antibodies can be used for defining the mechanism whereby Chd5 mediates expression of the Ink4/Arf locus. For example, these antibodies can be used to examine whether Chd5 directly binds the Ink4/Arf promoter using chromatin immunoprecipitation (ChIP).

### Example 15

### Overexpression of Chd5cDNA in Mammalian Cells

In order to examine the subcellular localization of Chd5 within the cell, tagged versions of Chd5 were expressed in mammalian cells. The complete cDNA for mouse Chd5 was cloned into a mammalian expression vector so that fluorescent labels (either mCherry or YFP) would be fused to the amino terminus of the Chd5 protein. These constructs were transfected into passage 3 mouse embryonic fibroblasts (mefs) and cells were analyzed 24-hours later. This analysis revealed that exogenously expressed Chd5 was nuclear, and that expression was particularly robust in rings surrounding chromocenters (regions of inactive transcription). This pattern of expression in regions associated with sites of actively transcribed chromatin demonstrates that Chd5 maintains chromatin in a transcriptionally competent state.

### Example 16

### Gene Targeting of the Chd5 Locus in Mouse Embryonic Stem (ES) Cells

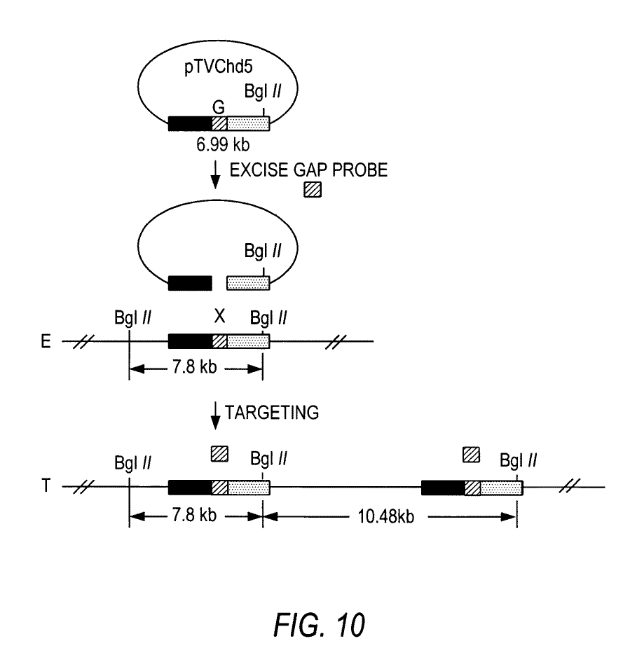

To assess tumorigenesis in mice lacking Chd5 specifically, we used gene-targeted disruption to inactivate the Chd5 locus in both +/+ and df/+ ES cells (FIG. 10). The resultant ES cells are used to generate Chd5 deficient mouse models and to monitor their susceptibility to tumorigenesis.

### Example 17

### Resequencing CHD5 in Human Tumors

To analyze CHD5's role in human cancers, the genomic DNA samples from human cancer cells are sequenced in the CHD5 locus (e.g., human cancers that have been shown to have CHD5 deletions (Bagchi et al., Cell 128:459-475 (2007)). This will provide an in-depth picture of the mutation spectrum of CHD5, including potential mechanisms involving non-exonic Cis-regulatory elements.

### Example 18

### The Role of Chd5 in Stem Cells

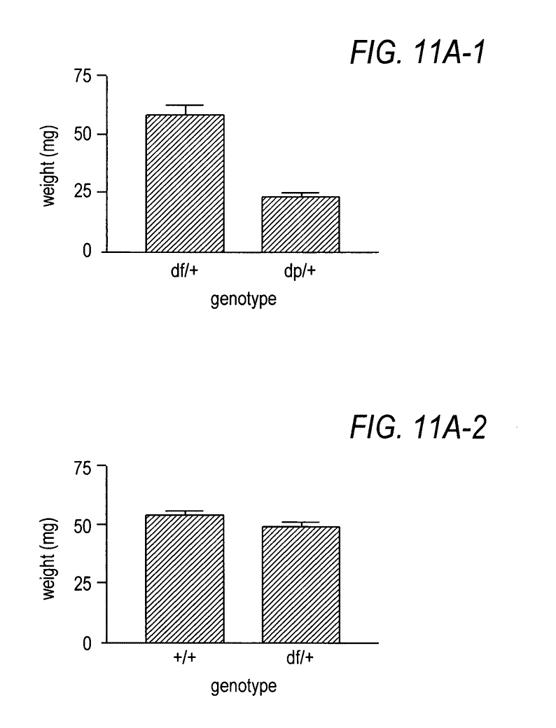

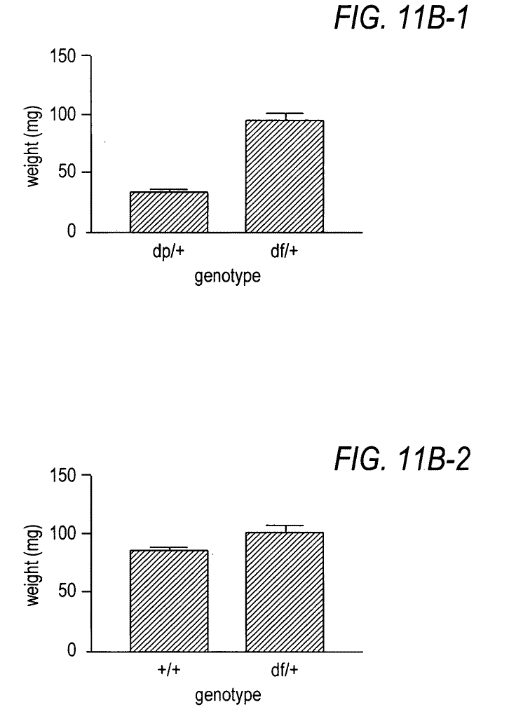

As shown above, Chd5 regulates the expression of the Ink4/Arf locus. This finding supports that Chd5 is a chromatin remodeling protein that serves as a master switch for a tumor suppressive network involving p16/Rb—and p19/p53-modulated pathways. We discovered that several organs of dp/+ mice were smaller than those of df/+ mice, including the fetal liver and brain (FIG. 11). This showed that excessive Chd5 caused a defect in stem cell renewal and/or differentiation.

Further, we established embryonic stem (ES) cells of the following genotypes: df/Chd5-null, df/+, +/+, and dp/+. Proliferation assays of these cells were done to analyze self renewal ability by plating the cells on gelatinized plates and growing them in the presence or absence of LIF for five days. The cells were then stained for alkaline phosphatase, where purple clones corresponded to self-renewing ES cells. The data showed that in the presence of LIF, the dp/+ ES cells had less self renewal ability than df/+ or df/Chd5 ES cells. When the same cells were allowed to differentiate in the absence of LIF, df/+ and df/Chd5 ES cells had more self-renewing potential than their wild type or dp/+ counterpart.

These data indicated that dosage of Chd5 correlated inversely with proliferative capacity. That is, dp/+ cells had the most severe compromise in proliferation, whereas df/Chd5-null ES cells had a significant increase in proliferation. Cells with enhanced dosage of Chd5 displayed a more differentiated phenotype, even in the presence of factors that maintain the pluripotent state. Thus, multiple types of stem cells were affected by Chd5, supporting the finding that a wide range of tumors developed in df/+ mice and that a diverse array of malignancies were associated with deletion of human 1p36.

### Example 19

### Experimental Procedures

A. Chromosome Engineering

Chromosome engineering was performed as previously described (Zheng et al., 2001, Methods 24, 81-94). Briefly, AB2.2 ES cells (from the129S5 strain) were maintained on irradiated SNL76/7 feeder cells and were used for gene targeting to sequentially modify the D4Mit190 and D4Mit51 endpoints using ˜25 μg of the pTVD4Mit190 and pTVD4Mit51 gene targeting insertion vectors. Clones were selected in G418 (180 μg/ml) or puromycin (3 μg/ml) for 8-10 days and targeting assessed by Southern analysis using the 1.5 kb HpaI or the 1.3 kb SacI gap probe, respectively. Doubly targeted clones were electroporated with ˜25 μg of the Cre-expressing plasmid pOG231 and grown in HAT (0.1mM sodium hypoxanthine, 0.2 μM aminopterin and 0.016 mM thymidine) to select for recombination between the integrated loxP sites. Cells were released from HAT selection by growing in HT (0.1 mM sodium hypoxanthine and 0.016 mM thymidine) for 48 hours and HAT-resistant clones were expanded and sib-selected in all possible combinations of HAT, G418, and puromycin to identify clones that were targeted in Trans in which Cre-mediated recombination occurred in G1. Triply-resistant dfldp clones were established as mouse strains as previously described.

B. Generation, Maintenance and Genotyping of Mouse Strains

Male chimeras were crossed to C57BL/6J females to generate F1 progeny including Agouti dfl+ and dp/+ embryos that were identified by PCR using the primers shown below and/or by Southern using the same strategy as for gene targeting. dfldp and p53+/− mice were intercrossed and Southern analysis of BamHI-digested DNA using a p53 cDNA probe identified endogenous and targeted p53 alleles as described previously (Donehower et al., supra). PCR was performed with 10 ng genomic DNA using a MASTERCYCLER™ gradient thermocycler (Eppendorf). Reaction products were resolved on 2-4% agarose gels. The following primers:

df FWD-

| | |
|---|---|
| 5′-CCTCATGGACTAATTATGGAC-3′; | (SEQ ID NO: 2) |
| df REV- | |
| 5′-CCAGTTTCACTAATGACACA-3′; | (SEQ ID NO: 3) |
| dp FWD- | |
| 5′-AACTGGCTGAGTGACGCCCTTTAT-3′; | (SEQ ID NO: 4) |
| dp REV- | |
| 5′-TGAGACGTGCTACTTCCATTTGTC-3′; | (SEQ ID NO: 5) |
| β-actin FWD: | |
| 5′-TGGAATCCTGTGGCATCCATGAAAC-3′; | (SEQ ID NO: 6) |
| β-actin REV: | |
| 5′-TAAAACGCAGCTCAGTAACAGTCCG-3′; | (SEQ ID NO: 7) |
| D4Mit308 FWD- | |
| 5′-TATGGATCCACTCTCCAGAAA-3′; | (SEQ ID NO: 8) |
| D4Mit308 REV- | |
| 5′-CAAAGTCTCCTCCAAGGCTG. | (SEQ ID NO: 9) |

C. Cellular Assays

Mefs were generated using standard procedures. Proliferation was assessed by plating 1×10⁵ mefs on multiple 6-well dishes, and harvesting and counting plates in triplicate at 2-day intervals using a Z1 Coulter Particle counter (Beckman-Coulter). Immortalization assays were performed by plating 2×10⁵ cells on triplicate plates and serially passaging and replating 2×10⁵ cells every 3 days. Graphing and standard error for both proliferation—and immortalization assays were done using Prism. Cell cycle analysis of mefs was performed by flow cytometry using a BD™ LSRII (BD Biosystems), and data was analyzed by FACS DiVa software (BD Biosystems). Cellular senescence was determined using an enzymatic assay for endogenous SA-β-gal as previously described. Foci formation assays were performed by growing cells at high density and counting the number of colonies that formed. Ras-infected cells were assayed for transformation by staining colonies in crystal violet and by assessing anchorage independent growth in soft agar. Soft agar assays were performed as previously described by plating serial dilutions of mefs in 0.4% agar and assessing the morphology and number of colonies.

D. Histological Analysis and TUNEL Assays

E17.5 dp/+ embryos and +/+ controls as well as multiple organs from adult dfl+ mice were harvested and processed as described previously (Keyes et al., 2005, Genes Dev 19, 1986-1999). Pathology was assessed by H.V. Unstained sections from dp/+ embryos and +/+ controls were analyzed for apoptosis using TUNEL (Amersham) according to the manufacturers protocol, and apoptotic cells detected by microscopy.

E. Design and Expression of shRNA

shRNAs (Table 2) were cloned in the MSCV-U6miR30-PIGΔRI (Dickins et al., 2005, Nat Genet 37, 1289-1295) with by-Ras expressing construct or with GFP (pMSCV-puro-PIG) (Hemann et al., 2003, Nat Genet 33, 396-400), as previously described (Keyes et al., supra). Retrovirus was produced by transiently transfecting the Phoenix ecotropic packaging cell line (G. Nolan, Stanford University) with shRNAs for 48 hours, the packaging cells were fed with media (DMEM, 10% FBS, 1% penicillin/streptomycin) for 24 hours, and mefs were infected by treatment with the viral supernatant for 12 hours. Cultures were fed with fresh media and after 48 hours were subjected to selection for 6-8 days.

TABLE 2
Target sequences for shRNAs

| Prdm16-a | |
|---|---|
| GCCCTCCTTGCATTATGCTAA | (SEQ ID NO: 10) |
| Prdm16-b | |
| CGGAAGAGCGTGAGTACAAAT | (SEQ ID NO: 11) |
| Kcnab2-a | |
| GGCAATAAACCCTACAGCAAA | (SEQ ID NO: 12) |
| Kcnab2-b | |
| GCCCTTTATCACTGGGATAAA | (SEQ ID NO: 13) |
| Icmt-a | |
| GGAAGAGTACTTGGATTATAA | (SEQ ID NO: 14) |
| Icmt-b | |
| TGCCCACAGGCCTGCCTTTCA | (SEQ ID NO: 15) |
| Hes3-a | |
| GGCACCACAACCTGCATCAAA | (SEQ ID NO: 16) |
| Hes3-b | |
| GGAGCTGAGTGTTAAGTACAT | (SEQ ID NO: 17) |
| Chd5-2 | |
| CCGAGATACATGATCCTAAAT | (SEQ ID NO: 18) |
| Chd5-4 | |
| CGCAAGCAGGTCAACTACAAT | (SEQ ID NO: 19) |
| Hes2-a | |
| CGAGAGCCTAAGCCAGCTGAA | (SEQ ID NO: 20) |
| Hes2-b | |
| CCGCTCTTCGAAGTTGGAGAA | (SEQ ID NO: 21) |
| Hkr3-a | |
| CCTCAGCAAATATTACCTGAA | (SEQ ID NO: 22) |
| Hkr3-b | |
| GGCGACACAAAGGTGTGAGAA | (SEQ ID NO: 23) |
| Dnajc11-a | |
| CCGCAGATTGAGATTAATAAA | (SEQ ID NO: 24) |
| Dnajc11-b | |
| CGGCGGCGGCTCCATTAACTT | (SEQ ID NO: 25) |
| Camta1-a | |
| GCCATCCTTATCCAGAGCAAA | (SEQ ID NO: 26) |
| Camta1-b | |
| CCAAGTAATGTGAATGCTAAT | (SEQ ID NO: 27) |
| Park7-a | |
| GCTCTCATCCCGGGTCTGTAT | (SEQ ID NO: 28) |
| Park7-b | |
| AGAATTTATCTGAGTCGCCTA | (SEQ ID NO: 29) |
| Errfi1-a | |
| GGCCAGCAAAGCCAGACTATA | (SEQ ID NO: 30) |
| Errfi1-b | |
| GGTTAGCTCAACGCATTATTA | (SEQ ID NO: 31) |

F. Gene Expression Analysis Using Real-Time PCR

Total RNA was isolated from mefs using Trizol reagent (Gibco). RT PCR was performed on 0.5 μg total RNA using a SUPERSCRIPT™ first strand cDNA synthesis kit (Invitrogen), and real-time PCR was performed according to manufacturers recommendations using a Peltier Thermal cycler (MJ Research) and a SYBR Green PCR kit (Applied Biosystems). Samples were analyzed in triplicate and expression was compared to that of β-actin. Primers:

| q β-actin FWD- | |
|---|---|
| 5′-GATCTGGCACCACACCTTCT-3′; | (SEQ ID NO: 32) |
| q β-actin REV: | |
| 5′-GGGGTGTTGAAGGTCTCAAA-3′; | (SEQ ID NO: 33) |
| qp21 FWD- | |
| 5′-TTGCACTCTGGTGTCTGAGC-3′; | (SEQ ID NO: 34) |
| qp21 REV- | |
| 5′-TCTGCGCTTGGAGTGATAGA-3′; | (SEQ ID NO: 35) |
| qMdm-2 FWD- | |
| 5′-TGAATCCTCCCCTTCCATCA-3′; | (SEQ ID NO: 36) |
| qMdm-2 REV- | |
| 5′-TCTCACGAAGGGTCCAGCAT-3′; | (SEQ ID NO: 37) |
| qDnajc FWD- | |
| 5′-AGCGCGAAGAGAGGAGACTA-3′; | (SEQ ID NO: 38) |
| qDnajc REV- | |
| 5′-CACTTCCGGACACATCTTCA-3′; | (SEQ ID NO: 39) |
| qChd5 FWD- | |
| 5′-GAGAGCCACCAGCACCTATC-3′; | (SEQ ID NO: 40) |
| qChd5 REV- | |
| 5′-TGCTTTCATGTCACTCAGCA-3′; | (SEQ ID NO: 41) |
| qp53 FWD- | |
| 5′-CGCTGCTCCGATGGTGAT-3′; | (SEQ ID NO: 42) |
| qp53 REV- | |
| 5′-TCGGGATACAAATTTCCTTCCA-3′; | (SEQ ID NO: 43) |
| qp16 FWD: | |
| 5′-GTCACACGACTGGGCGATT-3′; | (SEQ ID NO: 44) |
| qp16 REV: | |
| 5′-CATGCTGCTCCAGATGGCT-3′; | (SEQ ID NO: 45) |
| qp19 FWD- | |
| 5′-TGAGGCTAGAGAGGATCTTGAGA-3′; | (SEQ ID NO: 46) |
| qp19 REV- | |
| 5′-GCAGAAGAGCTGCTACGTGAA-3′. | (SEQ ID NO: 47) |

### G. Western Analysis

Immunoblotting was performed on mefs solubilized in Laemmli buffer and quantitated using a protein assay (Bio-Rad). Samples of 10-20 μg protein were analyzed using standard procedures, with antibodies for specific for Ras (mouse monoclonal, BD Transduction Laboratories, 1:500), p19 (Ab80, rabbit polyclonal, Abcam, 1:1000), p16 (M156, rabbit polyclonal, Santa Cruz, 1:500), p53 (CM5, Vector Laboratories, 1:500), and β-actin (AC-15, mouse monoclonal, Sigma, 1:10000). Secondary antibodies used were goat anti-mouse-HRP (IgG & IgM)—and goat anti-rabbit-HRP (IgG; Pierce, 1:5000).

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

What is claimed is:

1. A method of inhibiting growth of a mammalian cell, comprising increasing the level of a Chd5 protein within the cell.

2. A method of inducing apoptosis of a mammalian cell, comprising increasing the level of a Chd5 protein within the cell.

3. A method of inducing expression of p16^{Ink4a}, p19^{Arf}, or p53 in a mammalian cell, comprising increasing the level of a Chd5 protein within the cell.

4. The method of claim 1, 2, or 3, wherein said increasing comprises introducing to the cell a Chd5 protein.

5. The method of claim 4, wherein said introducing comprises contacting the cell with the Chd5 protein.

6. The method of claim 4, wherein said introducing comprises contacting the cell with an expression vector encoding the Chd5 protein.

7. The method of claim 6, wherein said expression vector is a viral vector.

8. The method of claim 1, 2, or 3, wherein the mammalian cell is a human, mouse, or nonhuman primate cell.

9. The method of claim 8, wherein the mammalian cell is a human cell.

10. The method of claim 8, wherein the mammalian cell is a cancer cell.

11. The method of claim 10, wherein the cancer cell is a human breast cancer cell, a human lymphoma cell, a human glioma cell, a human squamous cancer cell, a human sarcoma cell, or a human kibernoma cell.

12. The method of claim 11, wherein the mammalian cell has a deletion in the endogenous Chd5 allele.

13. The method of claim 10, wherein the mammalian cell is a cancer cell in a human patient.

14. A method of increasing growth of a mammalian cell, comprising reducing the Chd5 level in the cell.

15. The method of claim 14, wherein said reducing comprises introducing into the cell an antisense or interfering RNA against an endogenous Chd5 transcript.

16. The method of claim 14, wherein the mammalian cell is a human, mouse, or nonhuman primate cell.

17. The method of claim 16, wherein the mammalian cell is a stem cell.

18. A method of diagnosing cancer in a human subject, comprising:
providing a DNA sample from a tissue of the patient, and
detecting, in said DNA sample, deletion of (1) a nucleic acid sequence in a Chd5 gene, or (2) a nucleic acid sequence flanking the Chd5 gene in the D4Mit190-D4Mit51 interval in chromosome region 1p36, wherein said deletion indicates that the patient has, or is susceptible of developing, cancer in said tissue.

19. A method of diagnosing cancer in a human patient, comprising:
providing a tissue sample from the patient,
determining the level of a Chd5 protein in said tissue sample,
comparing said level of said Chd5 protein in the patient sample with a level of said Chd5 protein in a control sample, wherein a lower level of said Chd5 protein in said patient sample as compared to the control sample is indicative of the presence of cancer or susceptibility to cancer.

20. The method of claim 19, wherein said determining comprises contacting an extract of the tissue sample with an antibody that specifically binds to Chd5, said antibody being detectably labeled.

21. A mouse some or all of whose cells comprise a genome comprising an inactivating mutation in a Chd5 gene.

22. The mouse of claim 21, wherein said genome further comprises an activated oncogene.

23. A mammalian cell whose genome comprises an inactivating mutation in a Chd5 gene.

24. The mammalian cell of claim 23, wherein the genome further comprises an activated oncogene.

25. The mammalian cell of claim 23, wherein the cell is a human, mouse, or nonhuman primate cell.

26. A mouse some or all of whose cells comprise a genome comprising a coding sequence for an antisense or interfering RNA against a Chd5 transcript.

27. The mouse of claim 26, wherein the genome further comprises an activated oncogene.

28. The mouse of claim 26, wherein the coding sequence for the RNA is under the transcriptional control of an inducible promoter.

29. A mammalian cell whose genome comprises a coding sequence for an antisense or interfering RNA against a Chd5 transcript.

30. The mammalian cell of claim 29, wherein the genome further comprises an activated oncogene.

31. The mammalian cell of claim 29, wherein the coding sequence for the RNA is under the transcriptional control of an inducible promoter.

32. The mammalian cell of claim 29, wherein the cell is a human, mouse, or nonhuman primate cell.

33. A method of identifying an anti-cancer compound, comprising:
providing a mammalian cell,
contacting the cell with a candidate compound,
determining the level of Chd5 expression in the contacted cell,
wherein an increased level of Chd5 expression in the contacted cell as compared to a control cell indicates that the candidate compound has an anti-cancer effect.
